(19) United States
(12) Patent Application Publication
(10) Pub. No.: US 2024/0139126 A1
(43) Pub. Date: May 2, 2024

(54) NUTRITIONAL COMPOSITIONS FOR SKELETAL MUSCLE

(71) Applicant: MITOCHOLINE LTD, London (GB)

(72) Inventors: Larisa Andreeva, London (GB); Galina Nonina Skladtchikova, Hellemp (DK)

(21) Appl. No.: 18/394,525

(22) Filed: Dec. 22, 2023

Related U.S. Application Data
(63) Continuation-in-part of application No. 18/566,415, filed on Jan. 1, 0001.

(30) Foreign Application Priority Data
Jun. 3, 2021 (GB) 2107957.9

(51) Int. Cl. A61K 31/14; A23L 2/52; A23L 33/00; A23L 33/15; A23L 33/175; A61K 9/00; A61K 31/194; A61K 31/198; A61K 31/455; A61K 47/12; A61P 21/00

(52) U.S. Cl. CPC A61K 31/14; A23L 2/52; A23L 33/15; A23L 33/175; A23L 33/40; A61K 9/0095; A61K 31/194; A61K 31/198; A61K 31/455; A61K 47/12; A61P 21/00

(57) ABSTRACT

A method may formulate a composition to contain a predetermined dosage amount of choline and succinate in a molar ratio of choline to succinate of 2:1 that is effective to reduce a frequency and/or a severity of one or more symptoms selected from skeletal muscle weakness, skeletal muscle pain, low skeletal muscle tone, physical exercise intolerance, lack of muscular endurance, and loss of skeletal muscle mass in a human subject consuming the composition during an initial course of treatment. A method may provide to the human subject, the composition containing the predetermined dosage amount of choline and succinate in the molar ratio of choline to succinate of 2:1. In some examples, the composition may be formulated to increase skeletal muscle mass. In some examples composition may further include a nicotinamide component and/or a creatine component.

### CROSS-REFERENCES TO RELATED APPLICATIONS

The present application is a continuation-in-part of U.S. application Ser. No. 18/566,415 titled “NUTRITIONAL COMPOSITIONS FOR SKELETAL MUSCLE” and filed Dec. 1, 2023, which is a U.S. National Stage Entry of and claims the priority to PCT/GB2022/051316 titled “NUTRITIONAL COMPOSITIONS FOR SKELETAL MUSCLE” and filed on May 25, 2022, which in turn claims priority to UK application GB 2107957.9 filed on Jun. 3, 2021, each of which is incorporated herein by reference to the extent permitted by applicable patent law and rules.

### FIELD

This disclosure relates generally to compositions and methods for enhancing skeletal muscle.

### BACKGROUND

Achieving peak physical performance has long been a goal for athletic competition and self-improvement. Means for improving physical performance includes prolonged systematic exercise and proper diet. Incorporating good dietary practices, as part of an exercise program can optimize a human subject's performance during exercise.

### SUMMARY

In some aspects, the techniques described herein relate to a method for treating one or more symptoms in a human subject experiencing an abnormal skeletal muscle condition, the method including: formulating a composition to contain a predetermined dosage amount of choline and succinate in a molar ratio of choline to succinate of 2:1 that is effective to reduce a frequency and/or a severity of one or more symptoms selected from skeletal muscle weakness, skeletal muscle pain, low skeletal muscle tone, physical exercise intolerance, lack of muscular endurance, and loss of skeletal muscle mass in a human subject consuming the composition during an initial course of treatment; and providing to the human subject, the composition containing the predetermined dosage amount of choline and succinate in the molar ratio of choline to succinate of 2:1.

In some aspects, the techniques described herein relate to a method, further including providing the predetermined dosage amount of the composition for consumption by the human subject in a selected dosage form that includes from about 100 mg to about 1000 mg of choline and succinate in a molar ratio of choline to succinate of 2:1 per serving in one or more servings up to a total of about 2000 mg per day.

In some aspects, the techniques described herein relate to a method, further including, formulating the composition to further include: at least one nicotinamide component selected from nicotinamide, nicotinamide riboside, and/or nicotinamide mononucleotide.

In some aspects, the techniques described herein relate to a method, further including formulating the molar ratio of the choline to the succinate to the at least one nicotinamide component in the composition to be about 2:1:0.01-10.

In some aspects, the techniques described herein relate to a method, further including formulating the composition to further include at least one creatine component selected from creatine, arginine, and/or glycine.

In some aspects, the techniques described herein relate to a method, further including providing an initial treatment course that includes at least a five-day to seven-day supply of the composition in the selected dosage form.

In some aspects, the techniques described herein relate to a method, further including formulating the selected dosage form of the composition as a component of a medical food product or a medical beverage product.

In some aspects, the techniques described herein relate to a method, further including the selected dosage form of the composition as a component of a nutritional product.

In some aspects, the techniques described herein relate to a method, wherein the human subject is an aging human over 35 years old.

In some aspects, the techniques described herein relate to a method, wherein the abnormal skeletal muscle condition of the human subject is induced by a physical injury, a psychological factor, or an environmental factor.

In some aspects, the techniques described herein relate to a method, wherein the abnormal skeletal muscle condition is selected from muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, and/or sporadic inclusion body myositis.

In some aspects, the techniques described herein relate to a method for increasing skeletal muscle mass in a human subject, the method including: formulating a composition to contain a predetermined dosage amount of choline and succinate in a molar ratio of choline to succinate of 2:1 that is effective to reduce a frequency and/or a severity of one or more symptoms selected from skeletal muscle weakness, skeletal muscle pain, low skeletal muscle tone, physical exercise intolerance, lack of muscular endurance, and loss of skeletal muscle mass in a human subject consuming the composition during an initial course of treatment; and providing to the human subject, the composition containing the predetermined dosage amount of choline and succinate in the molar ratio of choline to succinate of 2:1.

In some aspects, the techniques described herein relate to a method, further including providing the predetermined dosage amount of the composition for consumption by the human subject in a selected dosage form that includes from about 100 mg to about 1000 mg per serving in one or more servings up to a total of about 2000 mg per day.

In some aspects, the techniques described herein relate to a method, further including providing an initial treatment course that includes at least a five-day to seven-day supply of the composition in the selected dosage form.

In some aspects, the techniques described herein relate to a method, further including formulating the composition to further include at least one nicotinamide component selected from nicotinamide, nicotinamide riboside, and/or nicotinamide mononucleotide.

In some aspects, the techniques described herein relate to a method, further including further the molar ratio of the choline to the succinate to the at least one nicotinamide component in the composition to be about 2:1:0.01-10.

In some aspects, the techniques described herein relate to a method, further including formulating the composition to further include at least one creatine component selected from creatine, arginine, and/or glycine.

In some aspects, the techniques described herein relate to a method, further including providing the composition as a sports beverage for consumption by a healthy human subject, a physically active human subject, and/or a person engaged in sports.

In some aspects, the techniques described herein relate to a method, further including formulating the dosage form of the composition as a component of a nutritional beverage or a sports beverage.

In some aspects, the techniques described herein relate to a method, further including formulating the dosage form of the composition for consumption by an aging human subject, or a human subject affected by a skeletal muscle loss as a symptom of a musculoskeletal disease or a musculoskeletal disorder.

In some aspects, the techniques described herein relate to a method, wherein the musculoskeletal disease or musculoskeletal disorder is selected from muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, sporadic inclusion body myositis.

### BRIEF DESCRIPTION OF THE FIGURES

In order that the advantages of the present disclosure will be readily understood, a more particular description of the present disclosure briefly described above will be rendered by reference to specific examples that are illustrated in the appended drawings. Understanding that these drawings depict only typical examples of the present disclosure and are not therefore to be considered to be limiting of its scope, the present disclosure will be described and explained with additional specificity and detail through the use of the accompanying drawings, in which:

FIG. 1A shows representative results of evaluation of NADH autofluorescence in primary myotubes after treatment with a selected DiSu+NAM composition, according to one or more examples of the present disclosure;

FIG. 1B shows representative results of a mitochondrial redox index in primary myotubes (which corresponds to NADH-dependent mitochondrial respiration) after treatment with a control, selected DiSu+NAM compositions, and individual constituents, according to one or more examples of the present disclosure;

FIG. 1C shows representative results of an evaluation of a mitochondrial pool of NADH (measured as a difference Δ between FCCP and NaCN NADH autofluorescence) after treatment with a control, selected DiSu+NAM compositions, and individual constituents, according to one or more examples of the present disclosure;

FIG. 2 shows representative results of an evaluation of mitophagy in primary cultures of myotubes after treatment with a control and after treatment with a selected DiSu+NAM composition evaluated as the level of co-localization of mitochondria and lysosomes, according to one or more examples of the present disclosure;

FIG. 3A shows representative results of evaluation of FAD autofluorescence following a treatment of cultures of primary myotubes with a selected DiSu+NAM composition, according to one or more examples of the present disclosure;

FIG. 3B shows representative results of an evaluation of a mitochondrial pool of FAD after treatment with a selected DiSu+NAM composition, as compared to a control treatment, according to one or more examples of the present disclosure;

FIG. 4 shows representative results of an evaluation of time to collapse of primary myotubes after treatment with treatment with a selected DiSu+NAM composition as compared to control treatment, according to one or more examples of the present disclosure;

FIG. 5A shows representative results of an evaluation of energy capacity of primary myotubes using a dexamethasone-induced sarcopenia cell culture model in which the inhibitor treated cells were maintained without any additional treatment (control), according to one or more examples of the present disclosure;

FIG. 5B shows representative results of a fluorescence evaluation of energy capacity of primary myotubes using a dexamethasone-induced sarcopenia cell culture model after treatment with a selected DiSu+NAM composition, according to one or more examples of the present disclosure;

FIG. 5C shows representative results of a fluorescence evaluation of energy capacity of primary myotubes using a dexamethasone-induced sarcopenia cell culture model after treatment with a selected DiSu+NAM composition, according to one or more examples of the present disclosure;

FIG. 5D shows representative results of an evaluation of time to collapse of primary myotubes after no treatment as compared to after treatment with a selected DiSu+NAM composition, according to one or more examples of the present disclosure;

FIG. 6A shows representative results of an in vivo evaluation of primary myotubes of young animals after treatment with a control as compared to after treatment with a selected DiSu+NAM composition, according to one or more examples of the present disclosure; and

FIG. 6B shows representative results of an in vivo evaluation of primary myotubes of aging animals after no treatment as compared to after treatment with a selected DiSu+NAM composition, and with another selected DiSu+NAM composition with a lower NAM molar ratio, according to one or more examples of the present disclosure.

### DETAILED DESCRIPTION

Reference throughout this specification to “one example,” “an example,” or similar language means that a particular feature, structure, or characteristic described in connection with the implementation is included in at least one example of the present disclosure. Thus, appearances of the phrases “in one example,” “in an example,” and similar language throughout this specification may, but do not necessarily, all refer to the same example.

### Introduction

The inventors of the human subject matter of the disclosure have recognized that good skeletal muscle function plays an important role in maintaining normal health and in enabling good sports performance.

In the context of skeletal muscle structure and function, autophagy and, in particular, mitophagy play critical roles in maintaining muscle health, function, and adaptation to various stimuli. Concerning autophagy in skeletal muscle, autophagy plays a role in the maintenance and repair of skeletal muscle. Autophagy in skeletal muscle cells helps in the turnover of damaged proteins and organelles. This is especially important after muscle use or injury, as it allows for the removal of damaged components and their replacement with new ones. Mitophagy is crucial for sustaining a healthy pool of mitochondria needed for supporting both muscle function and muscle mass.

Exercise may induce autophagy, and, in particular mitophagy of skeletal muscle cells, appear to be an integral part of beneficial adaptations induced by long-term exercise training. These processes helps in removing damaged cellular components, contributing to muscle adaptation, endurance, and recovery post-exercise. However, the effects of exercise may vary among different subjects due to genetic factors, the nature of the exercise, and other factors such as age.

As a subject ages, autophagic activity in muscles can decline, contributing to the accumulation of damaged proteins and organelles. This decline can be associated with age-related muscle loss and weakness.

A related aspect of skeletal muscle, mitophagy in skeletal muscle is also important. Mitophagy is crucial for maintaining healthy mitochondria in muscle cells. By removing damaged or dysfunctional mitochondria, mitophagy prevents the build-up of oxidative stress and ensures efficient energy production, which is vital for muscle function.

Exercise, especially endurance training, increases the demand for energy and can induce mitochondrial stress. Mitophagy is upregulated in response, helping to remove damaged mitochondria and stimulate the production of new, more efficient ones.

Mitophagy also plays a role in muscle differentiation and adaptation to various stimuli, like exercise or environmental stress. It's involved in the remodeling of muscle tissue and the regulation of muscle fiber type composition.

Impaired mitophagy in skeletal muscles has been linked to various pathologies, including muscular dystrophies, age-related and physical injury-related muscle loss (sarcopenia), and metabolic disorders.

In summary, autophagy and mitophagy in skeletal muscle are essential for muscle maintenance, adaptation, and function. They help in coping with stress, damage, and energy demands, and their dysregulation can contribute to muscle-related diseases and age-related decline.

The inventors of the subject matter herein have recognized the need to address muscle atrophy in certain subjects. During periods of muscle disuse or systemic conditions like fasting, cancer, or diabetes, autophagy can become upregulated, leading to increased degradation of muscle components. This is a double-edged sword; while it provides necessary nutrients and energy under starvation, it can also contribute to muscle atrophy.

For example, the continual supply of ATP to the fundamental cellular processes that underpin skeletal muscle contraction during exercise is essential for sports performance in events lasting seconds to several hours. Because the muscle stores of ATP are small, metabolic pathways must be activated to maintain the required rates of ATP resynthesis. These pathways include phosphocreatine and muscle glycogen breakdown, thus enabling substrate-level phosphorylation (‘anaerobic’) and oxidative phosphorylation by using reducing equivalents from carbohydrate and fat metabolism (‘aerobic’). The relative contribution of these metabolic pathways is primarily determined by the intensity and duration of exercise. The energy required to support long and intense exercise, e.g., a marathon run, is mainly provided through oxidative phosphorylation in the mitochondria of the active muscles.

Succinate plays a pivotal role in oxidative metabolism in general, and in skeletal muscle specifically. Succinate is a tricarboxylic acid (TCA) cycle intermediate that interacts directly with the mitochondrial electron transport chain (ETC), enabling a ‘shortcut1 route to ATP production via oxidative metabolism. Dietary succinate (sodium salt of succinic acid) supplementation increased endurance exercise ability in mice.

Nicotinamide (NAM), a form of vitamin B3, is a precursor of nicotinamide adenine dinucleotide (NAD), which is a redox-active metabolite. The depletion of NAD has been associated with body tissues aging and degenerative diseases. It has been reported that NAM administered to adult onset mitochondrial myopathy patients in the amount of 750-1, (X)0 mg/day increased in all human subjects both blood and muscle NAD* levels, muscle strength and mitochondrial biogenesis.

Certain inventors of the present human subject matter have also demonstrated that formulations of succinate (in a form of dicholine salt) and nicotinamide are synergistically effective for increasing the levels of both NAD+ and adenosine triphosphate (ATP) in the brain cells. Interestingly, these formulations also contain choline as described in WO2019002858.

Choline plays a main role in many physiological pathways, including the synthesis of neurotransmitters (acetylcholine). The signaling of the cell membrane (phospholipids) and lipid transport (lipoproteins) among others. Endurance exercise may enhance some of these pathways, increasing the choline demand as a metabolic substrate. According to some research data, choline affects skeletal muscle by modulating fat and protein metabolism, inflammation, and autophagy.

Currently available nutritional compositions for consumption before, during and after exercise typically comprise protein lysates or combination of amino acids to restore muscles fiber injuries caused by prolonged exercising and mixtures of different carbohydrates to fill the depleted carbohydrate energy stores of the body leading to reduced exercise performance.

However, existing compositions are not formulated to enhance oxidative phosphorylation in skeletal muscle, increase autophagy processes, and support physiological adaptation of the muscle mass to a prolonged exercise by supporting the conversion of skeletal muscle fiber from fast twitch to slow twitch induced by endurance or aerobic exercise.

Accordingly, the inventors of the human subject matter disclosed herein have developed various aspects of compositions and methods including the following.

A first aspect of the present disclosure relates to a composition comprising, or consisting essentially of, choline and succinate in the molar ratio choline to succinate from about 0.5:3 to about 3:0.5, preferably, about 2:1, for maintaining, enhancing or restoring energy metabolism and/or increasing autophagy and/or supporting physiological adaptation of the skeletal muscle mass to prolong physical exercise. Advantageously, the composition may also comprise nicotinamide (NAM), or a NAM derivate, such as nicotinamide riboside, wherein the molar ratio choline:succinate:NAM/NAM derivate, is about 0.5-3:3-0.5:0.01-10, e.g., about 1:2:0.01-10, preferably, 2:1:0.01-10, such as about 2:1:0.1-1. In some examples, the composition may consist essentially of choline cation, succinate anion (2-) and NAM or a NAM derivate in said molar ratio.

A second aspect of the present disclosure relates to a composition comprising, or in some examples. consisting essentially of choline and succinate, wherein the molar ratio choline:succinate is from about 0.5:3 to about 3:0.5, e.g., about 1:2, preferably, about 2:1, and, optionally, nicotinamide or a nicotinamide derivate, wherein the molar ratio choline:succinate:NAM/NAM derivate is about 0.5-3:3-0.5:0.01-10, e.g., 2:1:0.01-10, such as about 2:1:0.1-1, for improving physical performance in a human subject, requiring an enhanced skeletal muscle strength, muscle tone and endurance.

A third aspect of the present disclosure relates to a composition comprising, preferably consisting essentially of choline, succinate and NAM/NAM derivate, wherein the molar ratio choline:succinate:NAM/NAM derivate is about 0.5-3:3-0.5:0.01-10, e.g., 2:1:0.01-10, such as about 2:1:0.1-1, preferably consisting essentially of choline cation, succinate anion (2-) and NAM or a NAM derivate in said molar ratio, for use in maintaining or enhancing the skeletal muscle energy metabolism in an aging human, or in a human subject having or recovering from a disease.

A third aspect of the present disclosure relates to a composition comprising, or consisting essentially of, choline and succinate in the molar ratio 0.5-3:3-0.5, e.g., between 1:2 to 2:1, preferably, 2:1, and, advantageously, NAM, or a NAM derivate, wherein the molar ratio choline:succinate:NAM/NAM derivate is about 0.5-3:3-0.5:0.01-10, e.g., 2:1:0.01-10, such as about 2:1:0.1-1, preferably consisting essentially of choline cation, succinate anion (2-) and NAM or a NAM derivate in said molar ratio, for use in the dietary management of one or more symptoms and conditions associated with an imbalanced, damaged or reduced skeletal muscle energy metabolism and/or autophagy, such as muscle weakness, muscle pain, low muscle tone, exercise intolerance, lack of muscular endurance and loss of muscle mass.

A fourth aspect of the present disclosure relates to a method for maintaining, improving, or restoring skeletal muscle strength, tone and muscular endurance in a human subject, comprising administering to the human subject a composition consisting essentially of choline, succinate and, advantageously, NAM or NAM derivate, in a molar ratio from about 0.5-3:3-0.5:0.01-10, e.g., 2:1:0.01-10, such as about 2:1:0.1-1, wherein skeletal muscle of said human subject has a weakened or damaged mitochondrial function, or a weakened, unbalanced or damaged autophagy, preferably consisting essentially of choline cation, succinate anion (2-) and NAM or a NAM derivate in said molar ratio.

A fifth aspect, the present disclosure relates to a method for the dietary management of a symptom or condition associated with a musculoskeletal disease or disorder in a human subject, e.g., muscle-wasting, muscle degenerative disease, myopathies, age-related decline in muscle function, frailty, prefrailty, neuromuscular diseases, Duchenne muscular dystrophy, sarcopenia, muscle atrophy and/or cachexia, muscle loss, a muscle function disorder, age-related decline in muscle function, age-related sarcopenia, age-related muscle-wasting, physical fatigue, muscle fatigue, inclusion body myositis, imbalanced, damaged or reduced skeletal muscle energy metabolism in a human, or the dietary prevention of developing, occurring and/or re-occurring a symptom or condition associated with an imbalanced, damaged or reduced skeletal muscle energy metabolism in a human, comprising administering to said human, at least once a day, a composition comprising, choline, succinate in the molar ratio about 2:1, or, advantageously, comprising a composition comprising, choline, succinate and NAM, or a NAM derivate, in the molar ratio from about 0.5-3:3-0.5:0.01-10, e.g., 2:1:0.01-10, such as about 2:1:0.1-1, or in some examples, consisting essentially of choline cation, succinate anion (2-) and NAM or a NAM derivate in said molar ratio.

A sixth aspect of the present disclosure relates to a sport food or beverage for improving physical performance, increasing muscular strength and/or muscular endurance, comprising a composition comprising, or consisting essentially of, choline, succinate in the molar ratio about 0.5-3:3-0.5, e.g., from about 1:2 to 2:1, preferably, about 2:1. Advantageously, a composition may comprise, or consists essentially of, choline, succinate and NAM or a NAM derivate, in a molar ratio about 0.5-3:3-0.5:0.01-10, e.g., about 2:1:0.01-10, such as about 2:1:0.1-1.

In different examples, compositions of the present disclosure may be formulated as nutritional compositions, dietary supplements, functional or medical foods/beverages. The compositions are for the oral administration and may be administered in one or more doses daily for a period of one or more days. A single oral dose may contain from about 10 to about 5000 mg of a composition of the present disclosure.

Compositions of the present disclosure may further comprise additional ingredients, such as e.g., vitamins B9, B6, B12, or minerals such as Ca or Mg, or other ingredients, that are beneficial for maintaining and/or restoring the skeletal muscle healthy functioning and/or improving its performance.

A human subject who may benefit from the compositions of the present disclosure may be any human subject, including any healthy human subject with or without particular demand for improving his/her own physical performance, aging individuals and/or individuals who are weakened due to a disease or certain mental condition, such as e.g., psychological or environmental stress, fatigue, insomnia or mental depression.

Preferably, choline and succinate in compositions of the present disclosure are derived from dicholine succinate salt CAS No: 109438-15-5 (interchangeably, identified herein as choline succinate (2:1) salt or dicholine succinate or DiSu).

All terms and definitions explained throughout the text of specification relate to all aspects and implementations, unless otherwise specified.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C.

As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C.

As used herein, “a member selected from the group consisting of A, B, and C and combinations thereof” includes only A, only B, only C, a combination of A and B, a combination of B and C, a combination of A and C or a combination of A, B and C.

Support of skeletal muscle energy metabolism is of crucial importance both during intense physical activity and in case of muscle cell function/muscle mass decline associated with aging or disease. Well-functioning skeletal muscle energy metabolism is important for sustained and tonic contractile events, maintenance of energy homeostasis, and the alleviation of fatigue. There is a need for nutritional compositions capable of maintaining and increasing energy levels for an extended period of time, supporting muscle cells metabolome exhausted due to intensive training or due to disease or aging, and maintaining and enhancing the skeletal muscle fiber strength and endurance, and adaptability to prolonged exercise.

The present disclosure relates to synthetic compositions that are safe and effective to maintain, enhance and restore skeletal muscle energy metabolism in mammals in general, and in particular, in human subjects. The compositions can advantageously be used alone or in a combination with any diet and/or therapy to support or re-establish a proper skeletal muscle energy metabolism and to enhance the skeletal muscle strength and endurance in human subjects, including both physically active healthy human individuals and physically weakened individuals that are suffering or recovering from a disease of any age and in a combination with any diet and/or therapy.

Accordingly, the present disclosure in general relates to compositions that are effective to maintain, enhance and restore skeletal muscle energy metabolism, and maintain, enhance and restore physical performance in human subjects. Compositions of the present disclosure comprise at least two essential compounds, choline (cation) and succinate (anion 2-) in a molar ratio choline:succinate ranging from about 0.5:3 to about 3:0.5, such as from about 1:2 to about 2:1, preferably, about 2:1.

In some examples, the compositions additionally may comprise at least three compounds which are choline (cation) and succinate (anion 2-) and nicotinamide (NAM), or a NAM analog, e.g., nicotinamide riboside, wherein the molar ratio choline:succinate:NAM/NAM analog is about 0.5-3:3-0.5:0.01-10, preferably, about 2-1:1-2:0.01-10, e.g., 2:1:0.1-1.

It is surprisingly found that choline (cation) and succinate (anion 2-) in the indicated molar ratio, advantageously, in a combination with NAM, preferably, in the molar ratio choline:succinate:NAM about 2-1:1-2:0.01-10, e.g., 2:1:0.1-1, can synergistically enhance mitochondrial function of skeletal muscle cells and boost the production of “energy” molecules adenosine 5′-triphosphate (ATP), nicotinamide adenine dinucleotide (NAD) and FAD flavin adenine dinucleotide (FAD).

ATP is the main source of energy for all cellular processes. NAD and FAD are essential intracellular coenzymes that play key roles in cellular oxidation-reduction (redox) reactions and are responsible for accepting high-energy electrons and carrying them to the electron transport chain to synthesize ATP. The present inventors surprisingly found that a combination of choline and succinate, where the molar ratio of choline to succinate is from about 1-2:2-1 choline:succinate, and in some examples is 2:1, The present inventors found that the disclosed compositions strongly enhances the production of NAD, FAD and ATP in mitochondria of muscle cells.

The effect is even stronger, when such composition further comprises NAM, and the molar ratio choline:succinate:NAM is about 1-2:2-1:0.01-10, preferably 2:1:0.1-1. Preferably, choline and succinate of the composition are derived from dicholine succinate salt (DiSu), however, other salts of choline and succinic acid may be used as well.

None of components of the three-compound composition alone, nor in a combination of any two of the tree compounds were able to increase the production of said energy molecules to the same level as when both DiSu and NAM were present, still, a less profound effect of a combination of choline and succinate in the molar ratio choline:succinate from about 0.5:3 to about 3:0.5, such as from about 1:2 to about 2:1, preferably, about 2:1, was observed as well.

It is also found that oral administration of a composition consisting essentially of DiSu and NAM to human individuals for a period of time of one or more days leads to at least one of the following: an increased skeletal muscle strength, tone and endurance.

All terms and definitions explained throughout the specification of the present disclosure relate to all aspects and examples of the present disclosure, unless otherwise specified.

The wording “composition consisting essentially of” <named compounds> means that the named compounds of the composition are major compounds of the composition and contributing to the biological effect(s) associated with the composition.

The term “synthetic” in the present context means a man-made composition. The synthetic compositions of the present disclosure may comprise both synthetically prepared molecules that are structurally identical to the molecules that naturally occurring in the living bodies, and artificial molecules that do not have natural structural equivalents.

The terms “about” and “around” mean a deviation from the indicated value by 0.01% to 10%, such as from 0.5% to 5%.

The term “choline cation” (or “choline”) means the cation having the chemical formula C₅H₁₄NO⁺ (CAS No. 62-49-7).

The term “succinate anion” (or “succinate” or “succinate (2-)” means in the present context succinic acid anion (2-) having the chemical formula C₄H₄O₄⁻² (CAS No. 110-15-6), The terms “dicholine succinate”, “choline succinate salt (2:1)” and “DiSu” are interchangeable and mean the molecule of formula (I):

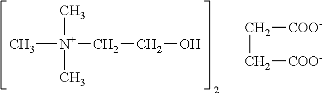

The term “nicotinamide” or “NAM” means the molecule identified with CAS No. 98-92-0. The term “derivate of nicotine amide” of NAM derivate” means a molecule that is derived from NAM by a synthetic process, e.g., NAM is a start molecule for the synthesis of the derivate such as nicotinamide riboside (CAS No: 1341-23-7) or nicotinamide mononucleotide (CAS No: 1094-61-7).

The present disclosure relates to compositions comprising, (in some examples, consisting essentially of) choline cation and succinate anion (2-); preferably the choline cation and succinate anion are present in the molar ratio about 0.5-3:3-0.5, preferably 2:1, preferably, said cation and anion are present in form of choline succinate salt (2:1) (DiSu), e.g., DiSu is a part of the composition. Alternatively, the choline cation of the composition may derive from another salt of choline, e.g., choline bitartrate (CAS No. 87-67-2), and succinate anion may derive from another salt of succinic acid, e.g., succinic acid disodium salt (CAS No. 6106-21-4).

Compositions consisting essentially of choline bitartrate, choline butyrate, choline chloride or choline fumarate, and succinic acid di-sodium or di-ammonium salt may be preferred in some examples of the present disclosure. The molar ratio of choline cation and succinate anion (2-) in such compositions is about 2:1.

Advantageously, a composition of the present disclosure comprising choline and succinate (molar ratio about 0.5-3:3-0.5, preferably, 2:1) comprises NAM or a NAM derivate. The amount of NAM, or a NAM derivate, in compositions of the present disclosure may vary within the range of molar ratio of choline to succinate to NAM/NAM derivate, e.g., the molar ratio may be within the range 0.5-3:3-0.5:00.1-10, such as from about 2:1:0.01 to about 2:1:10.

Compositions with a molar ratio of the individual compounds within this range act synergistically in enhancing mitochondrial function in skeletal muscle cells reflected by significant increase in the mitochondrial production of major energy molecules NAD, FAD and ATP.

These compositions are effective in dietary management of symptoms and conditions associated with imbalanced or weakened energy metabolism, weakened or damaged mitochondrial function and/or weakened or damaged autophagy in skeletal muscle cells, such as muscle weakness, muscle pain, low muscle tone, exercise intolerance, lack of muscular endurance and loss of muscle mass.

Furthermore, the compositions are beneficial as nutritional food supplements to improve physical performance of both healthy physically active human subjects and human subjects that are physically weaken due to aging, disease or psychological or environmental stress.

To obtain the above effects, the essential compounds of the composition, e.g., choline, succinate, and, advantageously, NAM, or a NAM derivate, are to be present in the compositions in an so-called “effective amount”. The effective amount of the compounds may vary depending on the aim and/or method of use, and/or human subject in need. These examples are discussed below and exemplified by non-limiting working examples.

In some examples, a composition of the present disclosure comprising choline cation, succinate anion (2-) and NAM may further comprise creatine (CAS No. 57-00-1), or a creatine precursor, such as e.g., amino acids glycine and arginine. Other useful additives to a composition comprising choline cation, succinate anion (2-) and NAM, or a NAM derivate, are discussed below.

Compositions of the present disclosure can be advantageously used for maintaining, enhancing and/or restoring a proper level of skeletal muscle energy metabolism in a human, wherein the term “proper level of skeletal muscle energy metabolism” means a dynamic capability of the skeletal muscle cells to generate energy by producing biological “energy” molecules NAD, FAD and ATP in the amounts that are sufficient to maintain optimal skeletal muscle functioning under normal conditions, including the muscle tone, strength of the contraction and the contraction endurance, and in the amounts that can compensate for an increased energy consumption under extraordinary conditions, e.g., such as an intense and/or prolonged physical exercise.

The term “imbalanced, damaged or reduced skeletal muscle energy metabolism” is synonymous with the term “improper level of skeletal muscle energy metabolism”, and the present content means that a dynamic capability of the skeletal muscle cells to generate energy by producing biological “energy” molecules NAD, FAD and ATP in the amounts that are sufficient to maintain optimal skeletal muscle functioning under normal conditions is weakened, e.g., due to a disease, aging, physical trauma, environmental or psychological stress, etc.

The proper level of skeletal muscle energy metabolism may differ from one human individual to another, and may depend on age, and/or physical capability of the individual. The proper level may be defined according to existing standards for physical performance, e.g., using physical performance studies described in Examples 3 and 4 of the specification.

The wording “maintaining skeletal muscle energy metabolism” means in the present context that compositions of the present disclosure provide support for the generation of energy molecules ATP, NAD and FAD in mitochondria to keep the energy metabolism in skeletal muscle of an individual on a proper level at any time.

The wording “restoring skeletal muscle energy metabolism” means in the present context that compositions of the present disclosure provide an extraordinary support in generation of energy molecules ATP, NAD and FAD by mitochondria of an energy-depleted and weakened skeletal muscle of an individual due to, e.g., a disease, aging, physical trauma, environmental or psychological stress factor etc. experienced or is experiencing by said individual, and helping thereby to said individual to overcome the energy exhaustion and restoration of the physical condition of the individual's skeletal muscle to a proper level.

The wording “enhancing skeletal muscle energy metabolism” means in the present context that compositions of the present disclosure, by supporting generation of energy molecules of ATP, NAD and FAD in mitochondria of skeletal muscle of a healthy individual, increase the energetic potential of skeletal muscle of said individual thereby allowing the individual's ability to efficiently use skeletal muscles over a longer period and under occasional stressful conditions, like an acute disease, physical trauma, psychological stress, etc.

Advantageously, for the maintaining, enhancing and/or restoring a proper level of the skeletal muscle energy metabolism, the compositions can be used by human individuals, both by healthy individuals and medical patients, as an everyday dietary supplement, as it is safe to use in a combination with any diet and therapeutic treatment. Advantageously, in one example, the compositions may be used for maintaining or, preferably, enhancing the skeletal muscle energy metabolism in an aging human.

The term “aging human” in the present context generally relates to a human individual over 24-25 years old, preferably over 34-35 years old, such as 30 to 45, about 50 or 60 years old or older. In one example, the composition can be advantageously used for the prophylaxis of aging of skeletal muscle. Skeletal muscle function declines and the muscle mass diminishes with aging.

Used on everyday basis for maintaining a proper skeletal muscle energy metabolism by a relatively young human individual, e.g., of 20-40 years old, compositions of the present disclosure have an advantageous capability to maintain the muscle mass and muscle strength and endurance at healthy levels through aging and enhance physical performance of said individuals for longer compared to individuals of the same aged who did not intake the same compositions. Thus, compositions of the present disclosure helps to decrease the speed of aging associated with the depletion of skeletal muscle mass and decline of skeletal muscle function.

As already mentioned above, human subjects according to present disclosure may be healthy physically active individuals, individuals suffering of or recovering from a disease, or individuals whose skeletomuscular activity is affected by a physical injury or psychological or environmental factors.

By “physical activity is meant any bodily movement produced by skeletal muscles that require energy expenditure. By “healthy physically active individual” in the present context is meant a human subject who has a good physical health, e.g., an individual that has his/her bodily functions and processes working normally, not necessarily at their peak, but not significantly deviating from their peak to affect individual complete physical, mental, and social well-being, in particular bodily functions and processes relating to musculoskeletal activity. In one preferred example, a healthy physically active individual is a human subject who has regular periods of intensive physical activity requiring significant energy expenditure, e.g., a human subject who is doing physical exercise to improve his/her physical performance such as a sportsman. Compositions of the present disclosure can help such individuals to restore, maintain and/or enhance the skeletal muscle energy metabolism and thereby to restore, maintain and/or enhance muscular tone, strength and endurance.

In another example, a healthy human subject is an aging human individual. The energy production and mitochondrial function decline in aging skeletal muscle cells, consequently skeletal muscle tone, strength and endurance decrease with age. The compositions of the present disclosure can be a beneficial supplement to the diet of aging human individuals, helping to their musculoskeletal system to overcome the age-related muscle function decline and thereby delay the onset of skeletal muscle aging. The compositions of the present disclosure are, surprisingly, effective in enhancing autophagy in skeletal muscle cells, which is essential for maintaining the muscle mass and healthy functioning skeletal muscle tissue. Therefore, compositions of the present disclosure may be useful for restoring, maintaining and/or enhancing skeletal muscle cell autophagy. Autophagy declines during muscle aging. Boosting basal autophagy of cells by promoting the selective degradation of misfolded proteins and dysfunctional organelles would protect the skeletal muscle from dysfunction induced and accelerated by aging.

Compositions of the present disclosure may also be advantageously used by individuals suffering of or recovering from a disease, or individuals affected by a physical injury or environmental or psychological stress factors. Often such individuals have a decreased level of energy production in all body cells, including skeletal muscle, and, in general, weakened skeletomuscular function and decreased physical activity. Accordingly, in one preferred example, the present disclosure relates to a human subject who is suffering of or recovering from a disease, or to an individual affected by a physical injury or an environmental or psychological stress factor, such as psychological pressure, fatigue, insomnia, mental depression, seasonal affective disorder (SAD), etc. The compositions of the present disclosure will boost energy production in skeletal muscle cells of these individuals and increase muscle tone, strength and endurance, helping thereby to these individuals to overcome their physical incapability.

In one example, the present disclosure relates to human subjects who are suffering of or recovering from a musculoskeletal disease, such as e.g., muscle degenerative diseases, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, sporadic inclusion body myositis. In one example, a human subject is an individual who is diagnosed with a metabolic myopathy, such as acid maltase deficiency (AMD, Pompe disease, glycogenosis type 2, lysosomal storage disease), carnitine deficiency, carnitine palmityl transferase deficiency (CPT deficiency), debrancher enzyme deficiency (Cori or Forbes disease, glycogenosis type 3), lactate dehydrogenase deficiency (glycogenosis type 11), myoadenylate deaminase deficiency, phosphofructokinase deficiency (Tarui disease, glycogenosis type 7), phosphogylcerate kinase deficiency (glycogenosis type 9), phosphogylcerate mutase deficiency (glycogenosis type 10), phosphorylase deficiency (McArdle disease, myophosphorylase deficiency, glycogenosis type 5). These diseases may be completely or partly overcome by adjusting diet to draw energy more efficiently from unaffected pathways. Compositions of the present disclosure could be a beneficial diet supplement contributing to recovery these diseases by adjusting, bypassing and/or enhancing energy channels in skeletal muscle cells of the affected human subjects.

Compositions of the present disclosure may be formulated as nutraceutical, nutritional or pharmaceutical compositions. These formulations will comprise effective amounts of the essential ingredients of the composition of the present disclosure in an appropriate molar ratio, as described above. The different formulations can be prepared according to standard rules and proceeding established in the corresponding art.

The term “nutraceutical” means a pharmaceutical-grade standardized nutrient. The term “pharmaceutical” in the present content means a pharmaceutical grade compound prescribed as medicament to treat a disease. The term “nutrient” means in the present context substance that provides nourishment essential for the maintenance of life of a human. The term “nutritional” in the present context means that the composition is for the dietary supplementation of a human individual. The term “dietary supplement” means a product taken by mouth that contains a dietary ingredient, e.g., a nutrient, intended to supplement the diet.

The amounts of choline cation, succinate anion (2-) and NAM, e.g., DiSu and NAM, in a composition of the present disclosure may be adjusted for use by a particular individual or a group of individuals according to the individual's needs, age, physiological conditions, etc., and depending on the dosage form and administration regime For example, the amount of NAM in the composition may vary from 4 mg to 4000 mg per serving, served as one or more dosages a day, such as about 20-2000 mg per serving, served as one or several dosages per day, or about 10-1000 mg per serving served as one or several dosages per day, etc., wherein the daily dose of NAM will depend on the dietary demand of a concrete human individual or a group of human individuals.

Non-limiting working examples of dietary compositions are described in the Examples described below. A daily intake of up to 4000 mg of NAM in the composition is considered safe and effective for any described herein purpose. The amount of DiSu per serving may vary from 10 mg to 1000 mg per serving, and it can be served in one or more dosages a day. According to the present disclosure, an individual may intake a composition comprising up to 4000 mg NAM, or a NAM derivate, and up to 1000 mg DiSu, or the corresponding amounts of choline cation and succinate (2-) anion derived from other salts of choline and succinic acid, daily without having any side effects.

In certain examples, a composition of the present disclosure is a nutritional composition and comprises essentially DiSu and NAM, wherein the molar ratio of choline cation, succinate anion (2-) and nicotinamide in the composition is about 2:1:0.1-1, such as about 2:1.:0.6-0.9 or about 2:1:0.2-0.5, e.g., about 2:1:0.4, correspondingly. The term “about” in the present context means a 1-10% deviation from the indicated value, e.g., the molar choline:succinate ratio indicated as 2:1 includes the molar ratios 1.7:0.9 or 2.3:1.1.

Preferably, intake of compositions of the present disclosure is continuous for a period of more than one day, preferably at least 5-7 days (one week) or, preferably, for a longer period, such as from 10-14 days to 20-30 days, or, preferably, longer, e.g., 2-3 months, 3-6 months, 12 months or longer. However, according to the present disclosure, a single intake of a composition of the present disclosure could also be beneficial for the skeletomuscular function of individuals, especially, if the individual is a sportsmen, and the composition is consumed right after said sportsmen' intense workout.

There is no limitation for how long the composition can be administered as a dietary supplement. In general, the longer intake of the composition leads to more pronounced beneficial effects. The intake can be interrupted at any time and resumed again when the individual feels that it is needed, e.g., in connection with changes in individual's lifestyle, health, individual's physical/mental conditions, or age. A dietary manager of ordinary skill can readily determine the amounts of ingredients of a dietary composition of the present disclosure according to the accepted rules and regulations.

As discussed above, dietary compositions of the present disclosure are useful for restoring, maintaining and/or enhancing muscular tone, strength and endurance in healthy and weakened human individuals. “Muscular tone” is defined herein as the tension in a muscle at rest. It is the muscle's response to an outside force, such as a stretch or change in direction. Appropriate muscle tone enables our bodies to quickly respond to a stretch. The term “muscular strength” means in general how much force a human individual can exert on a or how much weight a human individual can lift at once. The term “muscular endurance” refers to the ability of a muscle to sustain repeated contractions against resistance for an extended period of time.

Accordingly, use of a composition of choline cation, succinate anion (2-) and nicotinamide, or a nicotinamide derivate, as described herein, in a human, for restoring, maintaining and/or enhancing skeletal muscle energy metabolism, restoring, maintaining and/or enhancing muscular strength and endurance; maintaining and/or enhancing skeletal muscle function; treating a condition or a symptom associated with an imbalanced, damaged or reduced skeletal muscle energy metabolism, or reducing the risk of development or re-occurrence thereof;

In some examples, one or more compositions disclosed herein are useful for treating physical impairment associated with an imbalanced, damaged or reduced skeletal muscle energy metabolism, or reducing the risk of or delaying the onset thereof. Compositions comprising different combinations of choline cation, succinate anion (2-) and nicotinamide, or a nicotine derivate, as described herein, are beneficial and safe, and can be combined with a broad spectrum other nutritional compounds in beverages, nutritional supplements and foods or used alone without further additional ingredients.

The present disclosure also includes aspects relating to methods of dietary management of symptoms and conditions associated with imbalanced, damaged or reduced skeletal muscle energy metabolism, loss of balance due to muscle weakness and unstable joints, weakened muscle tone, strength and endurance, said methods comprising a step of administration of a composition according to the present disclosure to a human individual in need. The term “dietary management” in the present context means the practice of providing a nutritional option for individuals and groups with health concerns instead of a therapeutic intervention or as a prophylactic treatment, preferably under supervision of a dietary or medical professional.

Advantageously, the compositions of the present disclosure can be used for in dietary prophylaxis of primary and/or secondary development or occurrence of a symptom or condition associated with an imbalanced, damaged or reduced skeletal muscle energy metabolism in a human, including reducing the risk of occurrence and re-occurrence of the same symptom or condition, and reducing the strength and duration of the symptom or condition that is occurred for the first time or is re-occurring. The term “primary” means that the symptom or condition occurs in the human for the first time; the term “secondary” means that the symptom or condition is reoccurring.

Accordingly, a method for the dietary prevention of development, occurrence and/or re-occurrence of a symptom or condition associated with an imbalanced, damaged or reduced skeletal muscle energy metabolism in a human, wherein said symptom or condition may be any of the described herein, is one of the aspects of the present disclosure. The term “prevention” in the present context means mitigation of the risk or likelihood of occurrence and/or re-occurrence of a symptom or condition associated with an imbalanced, damaged or reduced skeletal muscle energy metabolism.

According to the present disclosure, compositions comprising certain ingredients described herein, e.g., choline, succinate and NAM, in the molar ratio of about 0.5-3:3-0.5:0.01-10, e.g., about 1:2:0.01-10, such as 2:1:0.01-10, in some examples about 2:1:0.1-1, provided for oral consumption to healthy individuals on a daily basis during at least 5-7 days, preferably for longer periods of time, are capable of creating a long-lasing increase in the energetic and renewal potential of the skeletal muscle cells allowing thereby the skeletal muscle cells functioning well and not deteriorate for longer.

Non-limiting examples of symptoms or conditions that could be advantageously relieved by the composition of the present disclosure are symptoms or conditions associated with musculoskeletal diseases and disorders, muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, sporadic inclusion body myositis or one or more metabolic myopathies described above.

The term wording “imbalanced, damaged or reduced” in the present context means that an individual's skeletal muscle energy metabolism is not on a proper level (as discussed above), but weakened by a disease, physiological, psychological or environmental condition, or aging of the individual.

As mentioned above, in some examples the present disclosure relates to nutritional compositions comprising, besides the essential compounds of the present disclosure (as discussed above), additional nutrients.

Examples of suitable additional ingredients include, but are not limited to, carriers, minerals, carbohydrates, lipids, vitamins, co-factors, buffers, flavors and sweeteners, inorganic salts, cations and anions typically abandoned in natural drinking water, taste modifying and/or masking agents, carbon dioxide, amino acids, organic acids, antioxidants, preservatives, and colorants.

The nutritional compositions can be combined with one or more carriers and used in the form of ingestible tablets, buccal tablets, troches, capsules, elixirs, suspensions, syrups, wafers, chewing gums, foods, beverages, and the like. Non-exclusive examples of ingredients which can serve as carriers include water; sugars, such as glucose, lactose, and sucrose; cellulose, and its derivatives; starches, such as corn starch and potato starch; powdered tragacanth; malt; gelatin; talc; excipients, such as cocoa butter; oils, such as olive oil, peanut oil, cottonseed oil, corn oil and soybean oil; glycols, such as propylene glycol; esters, such as ethyl oleate and ethyl laurate; polyols, such as glycerin, mannitol, sorbitol, and polyethylene glycol; agar; buffering agents; water; pH buffered solutions; and other non-toxic compatible substances employed in formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Non-exclusive examples of antioxidants are Vitamin E, ascorbic acid, carotenoids, aminoindoles, Vitamin A, uric acid, flavonoids, polyphenols, herbal antioxidants, melatonin, lipoic acids, and mixtures thereof.

Compositions of the present disclosure may additionally comprise useful inorganic salts, cations and/or anions. Non-exclusive examples of useful inorganic salts are sodium carbonate, sodium bicarbonate, potassium chloride, magnesium chloride, calcium chloride, and mixtures thereof. Non-exclusive examples of useful cations are sodium, potassium, magnesium, calcium, zinc, iron, and mixtures thereof. Non-exclusive examples of useful anions are fluoride, chloride, bromide, iodide, carbonate, bicarbonate, sulfate, phosphate, and mixtures thereof.

The pH of the liquid compositions could be adjusted to neutral using different buffers. Non-exclusive examples of suitable buffers are phosphate buffer, glycine buffer, citrate buffer, acetate buffer, carbonate buffer, tris-buffer, triethanolamine buffer, and succinate buffer.

Compositions may comprise some flavoring compounds, sweeteners and/or colorants. Non-exclusive examples of suitable flavors are synthetic flavor oils; flavoring aromatics and naturals oils such as cinnamon oil, oil of wintergreen, peppermint oils, clove oil, bay oil, anise oil, eucalyptus, thyme oil, cedar leave oil, oil of nutmeg, oil of sage, oils of citrus fruits, oil of bitter almonds, and cassia oil; plant extracts, flowers, leaves, fruits, vanilla, chocolate, mocha, coffee, apple, pear, peach, citrus such as lemon, orange, grape, lime, and grapefruit; mango, strawberry, raspberry, cherry, plum, pineapple, and apricot, and combinations thereof. Non-exclusive examples of suitable sweeteners are natural and synthetic sweeteners.

Non-limiting examples of natural sweeteners that may be utilized are naturally occurring substances, sucrose, extracts from naturally occurring substances; extracts of the plant Stevia rebaudiana compositae bertoni such as Stevia, steviol, rebaudiosides A-F, and dulcosides A and B; extracts of Thladiantha grosvenorii such as mogroside V and related glycosides and triterpene glycosides; phyllodulcin and its derivatives; thaumatin and its derivatives; mogrosides such as mogroside IV, mogroside V, siamenoside, and mixtures thereof; genus Siraitia including S. grosvenorii, S. siamensis, S. silomaradjae, S. sikkimensis, S. africana, S. borneesis, and S. taiwaniana; naturally-occurring glycosides; and active compounds of plant origin having sweetening properties, and mixtures thereof. Some examples of synthetic sweeteners are aspartame saccharin, and mixtures thereof. Certain examples of suitable colorants are dyes suitable for food such as those known as FD&C dyes, natural coloring agents such as grape skin extract, beet red powder, titanium dioxide, and beta-carotene, annatto, carmine, chlorophyll, paprika, and mixtures thereof.

In some examples, the compositions may include useful organic acids and/or amino acids. Non-exclusive examples of useful organic acids are acetic acid, butyric acid, malic acid, pyruvic acid, glutamic acid, citric acid, omega-3 unsaturated acids, linoleic acid, linolenic acid, eicosapentaenoic acid, docosahexaenoic acid, aspartic acid, and mixtures thereof. Non-exclusive examples of useful amino acids are Glycine, Arginine, L-Tryptophan, L-Lysine, Methionine, Threonine, Levocarnitine, and L-carnitine.

In various examples, the compositions may additionally comprise vitamins and/or cofactors. Non-exclusive examples of useful vitamins are thiamin, riboflavin, panthothenic acid, biotin, folic acid, pyridoxine, vitamin B12, lipoic acid, vitamin A, vitamin D, vitamin E, ascorbic acid, choline, carnitine; alpha, beta, and gamma carotenes; vitamin K, and mixtures thereof. Non-exclusive examples of useful co-factors are thiamine pyrophosphates, flavin mononucleotide, pyridoxal phosphate, biotin, tetrahydrofolic acid, Coenzyme A, coenzyme B12, 11-cis-retinal, 1,25-dihydroxycholecalciferol and mixtures thereof.

In one or more examples, a nutritional composition of the present disclosure may comprise compounds that are able to increase the blood circulation, e.g., an extract of Ginkgo biloba or ginseng. In some examples, a composition of the present disclosure may comprise an anti-oxidant, e.g., astaxanthin, resveratrol, flavonoids.

The above described and other beneficial optional ingredients generally are used at levels from about 0.0005% to about 10.0% by weight of a composition consisting of the essential components.

Such nutritional compositions of the present disclosure may be formulated as any type of nutritional product such as a food, a beverage, a dietary supplement, a functional food, and a medical food. In one or more examples, a composition of the present disclosure is an aqueous nutritional composition, e.g., a drink or beverage, such as a sport beverage. Certain examples of formulation of the compositions of present disclosure include sport nutritional supplements, sport foods and sport beverages. In one or more examples, a sport nutritional supplement, food or beverage comprises from about 10 mg to about 5000 mg of a composition consisting essentially of choline and succinate in the molar ratio of about 0.5-3:3-0.5, e.g., 1:2, preferably, 2:1.

In another example, a sport nutritional supplement, food or beverage comprises from about 10 mg to about 5000 mg of a composition consisting essentially of choline, succinate and nicotinamide, or a nicotinamide derivate, in the molar ratio, preferably, from about 2:1:0.01 to about 2:1:1. The other suitable molar ratios of the compounds are discussed throughout the specification. Preferably, choline and succinate are present in a sport nutritional supplement, food or beverage of the present disclosure the form of DiSu. A sport nutritional supplement, food or beverage of the present disclosure is useful for maintaining, improving or restoring physical performance, muscular strength and/or muscular endurance in a human subject who is spending time on physical exercise in order to improve his/her physical performance and increase the muscle mass.

In one example, a composition comprising from about 10 mg to about 5000 mg and consisting essentially of choline and succinate in the molar ratio about 2:1, or a composition comprising from about 10 mg to about 5000 mg and consisting of choline, succinate and nicotinamide, or a nicotinamide derivate, in the molar ratio from about 2:1:0.01 to about 2:1:10 may be advantageously used as a supplement to a ketogenic diet or the composition may be included in a ketogenic drink or food product. Elevated circulating ketone bodies from ketogenic diet used by skeletal muscle as a fuel alter substrate competition for respiration, while improving oxidative energy transduction under certain conditions, such as endurance exercise. Consequently, combination of compositions of the present disclosure with nutritional ketosis may help to unlock greater human metabolic potential, e.g., in endurance exercise.

In practicing the present disclosure, the compounds of compositions of the present disclosure can be prepared by any process known in the art or obtained from a commercial manufacturer, e.g., nicotinamide or its derivatives, choline bitartrate, succinate disodium salt, may be obtained from Merck. DiSu can be prepared by the reaction of choline hydroxide (CAS No. 123-41-1) with succinic acid (CAS No. 110-15-6) as, e.g., described in WO2009/022933A1. The nutritional compositions comprising the essential and, optionally, additional ingredients described herein, may be prepared by procedures well-known from the art.

In one example, a nutritional composition of the present disclosure can be used as a component of a food product. Nonexclusive examples of food products include regular foods, dietary supplements, beverages, and medical foods. The term “medical food” refers to a food which is formulated to be consumed or administered enterally under the supervision of a physician and which is intended for the specific dietary management of a disease, condition, or disorder.

Preferably, the nutritional compositions are formulated for oral administration and preferably administered to a human orally for a period of one or more days, preferably for at least 5-7 days or, even more preferably, for a longer period of time longer (as discussed above). However, human subjects will also benefit from a single oral administration of a composition of the present disclosure, e.g., human individuals who would intake the composition right after their physical workout. The compositions of present disclosure would help such individuals to more rapidly compensate for exhaustion of energy in their skeletal muscle by boosting the production of energy molecules in the skeletal muscle cells.

Non-limiting examples of compositions and their applications include:

A composition comprising choline and succinate in the molar ratio about 2:1, for maintaining, enhancing and/or restoring skeletal muscle energy metabolism, and/or autophagy in skeletal muscle in a human subject; preferably, the composition comprise, or consists essentially of choline, succinate and nicotinamide, or a nicotinamide derivate, in the molar ratio from about 2:1:0.01 to about 2:1:10. Preferably, the composition comprising choline succinate (2:1) salt. The nicotinamide derivate is preferably nicotinamide riboside. The amount of nicotinamide, or the nicotinamide derivate, is from about 10 mg to about 4000 mg, and the amount of choline succinate (2:1) salt is, preferably, from about 10 mg to about 1000 mg.

A composition comprising choline and succinate in the molar ratio about 2:1, for use in maintaining, improving, or restoring skeletal muscle tone strength and endurance in a human subject. The composition comprises, preferably, consists essentially of choline, succinate and nicotinamide, or a nicotinamide derivate, in the molar ratio from about 2:1:0.01 to about 2:1:10. The composition preferably comprises choline succinate (2:1) salt. The nicotinamide derivate is preferably nicotinamide riboside. The amount of nicotinamide, or the nicotinamide derivate, is from about 10 mg to about 4000 mg. The amount of choline succinate (2:1) salt is from about 10 mg to about 1000 mg. The composition is preferably a nutritional supplement, e.g., for an aging human subject, a physically active human subject. The composition is formulated as, e.g., a sport food, a sport beverage or a sport supplement.

The human subject is, in one example, a healthy human individual, in another example, a human subject who is suffering of or recovering from a disease, in another example, a human subject is an individual who is affected by a physical injury or a psychological or environmental factor. The composition, in one example, is for use in the dietary management of one or more symptoms and conditions associated with an imbalanced, damaged or reduced skeletal muscle energy metabolism, or dietary management of one or more symptoms and conditions associated with a weakened or damaged mitochondrion function in skeletal muscle, wherein the symptom or condition is preferably selected from muscle weakness, muscle pain, low muscle tone, exercise intolerance, lack of muscular endurance and loss of muscle mass.

A composition comprising choline and succinate in the molar ratio about 2:1, or a composition comprising, preferably, consisting essentially of choline, succinate and nicotinamide, or a nicotinamide derivate, in the molar ratio from about 2:1:0.01 to about 2:1:10 for a treating a musculoskeletal disease or musculoskeletal disorder in a human subject. The disease or disorder may be selected from muscle-wasting, muscle degenerative disease, myopathies, frailty, pre-frailty, neuromuscular diseases, Duchenne muscular dystrophy, sarcopenia, muscle atrophy and/or cachexia, muscle loss, a muscle function disorder, age-related decline in muscle function, age-related sarcopenia, age-related muscle-wasting, physical fatigue, muscle fatigue, inclusion body myositis, sporadic inclusion body myositis, or from metabolic myopathy, such as acid maltase deficiency (AMD, Pompe disease, glycogenosis type 2, lysosomal storage disease), carnitine deficiency, carnitine palmityl transferase deficiency (CPT deficiency), debrancher enzyme deficiency (Cori or Forbes disease, glycogenosis type 3), lactate dehydrogenase deficiency (glycogenosis type 11), myoadenylate deaminase deficiency, phosphofructokinase deficiency (Tarui disease, glycogenosis type 7), phosphogylcerate kinase deficiency (glycogenosis type 9), phosphogylcerate mutase deficiency (glycogenosis type 10), phosphorylase deficiency (McArdle disease, myophosphorylase deficiency, glycogenosis type 5). The composition is in one example nutritional and formulated as a food supplement, beverage, or food, such as a medical beverage or medical food; in another example, the composition is pharmaceutical.

A method for the dietary management of a condition or a symptom associated with an imbalanced, damaged or reduced skeletal muscle energy metabolism; or for the dietary mitigation of the risk of occurrence and/or re-occurrence of a symptom or condition associated with an imbalanced, damaged or reduced skeletal muscle energy metabolism in a human subject, comprising administering to said human at least once a day any composition of the present disclosure.

The symptom or condition is selected from muscle weakness, muscle pain, low muscle tone, exercise intolerance, lack of muscular endurance and loss of muscle mass. The composition may be administered daily in one or more doses for a period of one or more days.

Use of a combination of choline cation, succinate anion (2-) and nicotinamide, or a nicotine derivate in a human subject, for restoring, maintaining and/or enhancing skeletal muscle energy metabolism and/or mitochondrial autophagy; restoring, maintaining and/or enhancing muscular strength and endurance; maintaining and/or enhancing physical performance; treating or reducing the risk of development or re-occurrence of a condition or a symptom associated with an imbalanced, damaged or reduced skeletal muscle energy metabolism and/or autophagy; treating or reducing the risk of or delaying the onset of physical impairment associated with associated with an imbalanced, damaged or reduced skeletal muscle energy metabolism and autophagy, in the human subject; wherein the molar ratio of choline cation:succinate anion(2-):nicotinamide, or nicotinamide derivate, in the combination is from about 2:1:0.01 to about 2:1:10; preferably, wherein the combination comprising choline succinate (2:1) salt. Preferably, the combination is formulated as a nutraceutical or nutritional composition, e.g., the composition is formulated as a beverage or food product, such as a medical beverage or medical food product.

Various examples of a dosage form as a component of a nutritional supplement are listed below.

A sport nutritional supplement, food or beverage comprising from about 10 mg to about 5000 mg of a composition consisting essentially of choline and succinate in the molar ratio about 2:1.

A sport nutritional supplement, food or beverage comprising from about 10 mg to about 5000 mg of a composition consisting essentially of choline, succinate and nicotinamide, or a nicotinamide derivate, in the molar ratio from about 2:1:0.01 to about 2:1:10.

A sport nutritional supplement, food or beverage, wherein choline and succinate are present in the form of dicholine succinate salt.

A sport nutritional supplement, food or beverage of any of the above, for maintaining, improving or restoring physical performance, muscular strength and/or muscular endurance in a human subject.

### EXAMPLES

Non-limiting working examples are presented below to illustrate the human subject matter of the disclosure. The working examples are merely illustrative and should not be interpreted in any way as limiting the scope of the claims.

### Example 1

### Nutritional Compositions of the Present Disclosure

### Beverage-1

The beverage is prepared by mixing of NAM with DiSu in amounts as indicated below and dissolved in 330 ml of water to provide a beverage.

Beverage-1

| Ingredient | Content, per serving |
|---|---|
| NAM | 37 mg |
| DiSu | 250 mg |
| D-gluconic acid | qs to pH 6.5 |
| Water | to 330 ml |

The molar ratio choline:succinate:NAM in this beverage is 2:1:0.4).

### Beverage-2

The beverage is prepared by mixing of NAM with DiSu in amounts as indicated below and dissolved in 330 ml of water.

Beverage-2

| Ingredient | Content, per serving |
|---|---|
| NAM | 188 mg |
| DiSu | 250 mg |
| D-gluconic acid | qs to pH 6.5 |
| Water | to 330 ml |

The molar ratio choline:succinate:NAM in this beverage is 2:1:2.

### Beverage-3

The beverage is prepared by mixing of NAM with DiSu in amounts as indicated below and dissolved in 330 ml of water.

Beverage-3

| Ingredient | Content, per serving |
|---|---|
| NAM | 188 mg |
| DiSu | 500 mg |
| D-gluconic acid | qs to pH 6.5 |
| Water | to 330 ml |

The molar ratio choline:succinate:NAM in this beverage is 2:1:1.

### Beverage-4

The beverage is prepared by mixing of NAM with DiSu in amounts as indicated below and dissolved in 500 ml of water.

Beverage-4

| Ingredient | Content, per serving |
|---|---|
| NAM | 210 mg |
| DiSu | 560 mg |
| D-gluconic acid | qs to pH 6.5 |
| Water | to 500 ml |

The molar ratio choline:succinate:NAM in this beverage is 2:1:1.

### Beverage-5

The beverage is prepared by mixing NAM with DiSu in amounts as indicated below and dissolved in 330 ml of water.

Beverage-5

| Ingredient | Content, per serving |
|---|---|
| NAM | 375 mg |
| DiSu | 100 mg |
| D-gluconic acid | qs to pH 6.5 |
| Water | to 330 ml |

The molar ratio choline:succinate:NAM in this beverage is about 2:1:10.

### Beverage-6

The beverage is prepared by mixing of NAM with DiSu in amounts as indicated below and dissolved in 330 ml of water.

Beverage-6

| Ingredient | Content, per serving | |
|---|---|---|
| NAM | 7 | mg |
| DiSu | 2000 | mg |

| | |
|---|---|
| Succinic acid | qs to pH 6.5 |
| Water | to 330 ml |

The molar ratio choline:succinate:NAM in this beverage is 2:1:0.01

### Beverage-7

The beverage is prepared by mixing of NAM with DiSu in amounts as indicated below and dissolved in 330 ml of water to provide a beverage.

Beverage-7

| Ingredient | Content, per serving |
|---|---|
| NAM | 37 mg |
| DiSu | 250 mg |
| Citric acid | qs to pH 6.5 |
| Water | to 330 ml |

The molar ratio choline:succinate:NAM in this beverage is 2:1:0.4.

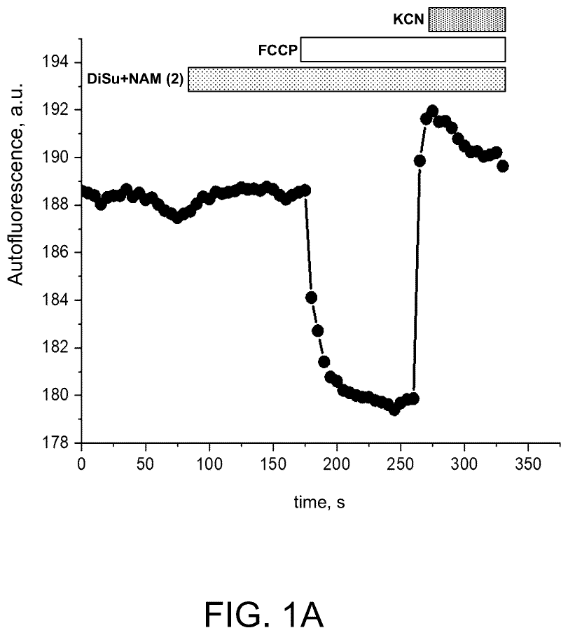

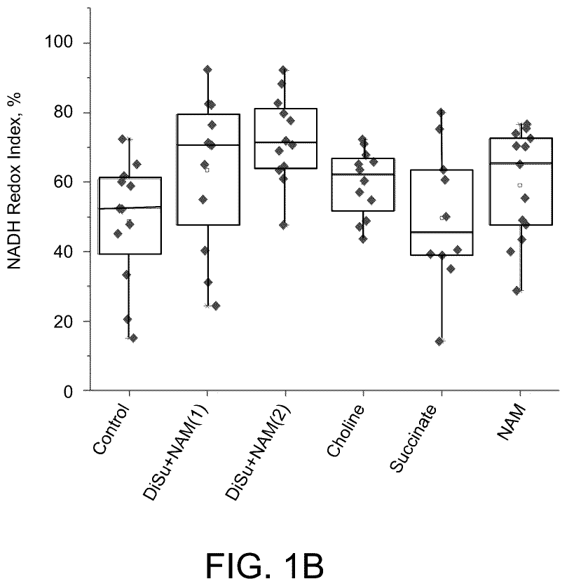

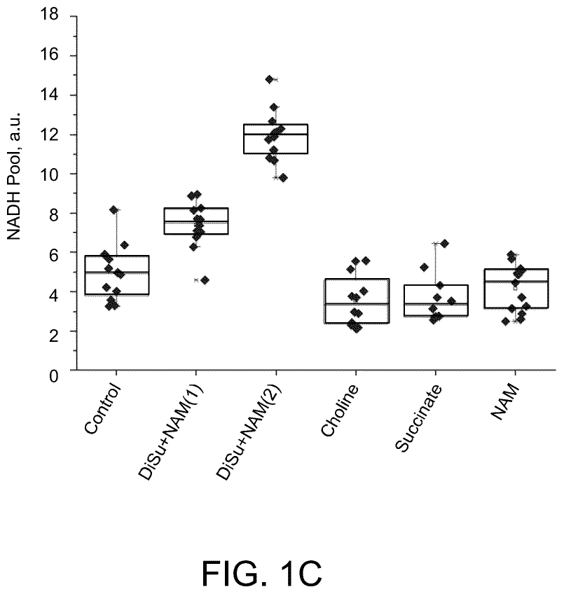

FIGS. 1A-1C shows representative results of an various intermediate and clinical effects on skeletal muscle cell function of compositions containing choline and succinate in a molar ratio of 2:1 such as disclosed above. More details concerning the results depicted in FIGS. 1A, 1B and 1C are described in the following sections.

### Example 2. Evaluation of the Effect of Compositions of the Present Disclosure on Cellular Energetics and Mitophagy in Primary Cultures of Myocytes

### Materials and Methods.

Primary Skeletal Muscle Cell culture. Primary myotube cells were obtained from postnatal day 0-2 (P0-P2) rat pups which were sacrificed by decapitation. Briefly, the rats were immersed in 70% ethanol and the hind-limbs removed under a dissection microscope, in dissection solution containing Dulbecco's Phosphate Buffered Saline (PBS) with penicillin streptomycin (5000 units/ml) and Amphotericin B (2.5 mg/L). The tissues were then digested in 0.2% collagenase solution at 37° C. for 30-40 minutes. The reaction was stopped by adding 2-3 ml fetal bovine serum (FBS), and the tissue suspension was centrifuged at 2010 rpm (700 g) for 10 minutes. The cell pellet was resuspended in pre-warmed growth medium, Dulbecco's modified Eagle's medium (DMEM), containing 20% FBS, 100 i.u./ml penicillin and 200 i.u./ml streptomycin], and plated on 22 mm coverslips that were pre-coated with 0.2% gelatin for 1 hour at 37° C. Cells were then cultured in a humidified CO2 incubator (5% CO2 in air) at 37° C. After 2 or 3 days, the medium was changed to a low-serum medium (DMEM containing 10% FBS without antibiotics), and was subsequently renewed every 3 or 4 days. All experiments were performed in vitro between days 2 and 11 in culture when spindle-shaped myoblasts had proliferated and aligned themselves for fusion (˜70% confluency).

Effect of compositions of the present disclosure on capacity of primary myocyte (myotubes) mitochondria to produced “energy” molecules ATP, NAD and FAD was assessed in resting cells and activated cells in the cellular model of exercise (myotubes contractions are induced by different caffeine concentrations, as described below).

In particular, the effect of acute application of the compounds (e.g., dicholine succinate (DiSu) and nicotinamide (NAM) and a combination thereof) on mitochondrial membrane potential, level of mitochondrial NADH and FAD, and intracellular ATP was studied on resting myotubes using a 24- and 48-hours incubation of the myotubes with the compounds.

Myotubes activated by:
1 mM caffeine to induce singe calcium peaks and myotube contractions to simulate ‘physiological’ level of myotube activity resembling the activity of exercise,
5 mM caffeine to stimulate a near maximal [Ca2+]c signal and fast contractions of myotubes, and
10 mM caffeine to cause potentially damaging [Ca2+]c overload,
were used as an “exercise model”. The cell cultures are incubated with the compounds to evaluate the effect on the level of production of ATP, NAD, FAD and mitochondrial membrane potential during exercise.

Confocal Microscopy. Confocal images were obtained using an inverted Zeiss 710 UV-Vis CLSM equipped with a META detection system and a 40× oil immersion objective. The 488 nm Argon laser line was used to excite fluo-4, MagFluo-4 fluorescence, which were all measured using a bandpass filter of 505-550 nm. Illumination intensity was kept to a minimum (at 0.1-0.2% of laser output) to avoid phototoxicity and the pinhole set to give an optical slice of ˜2 μm.

NADH and its oxidized form NAD act as hydrogen carriers at the site of the electron transport chain during mitochondrial respiration. The fluorescent properties of NADH make it a valuable fluorescent indicator of the mitochondrial metabolic state. NADH autofluorescence was excited at 351 and measured at 375-470 nm.

Tetramethyl rhodamine methyl ester (TMRM) was used to study the changes of the mitochondrial membrane potential (Δψₘ). Cells were loaded with 25 nM TMRM for 40 min until a steady state was achieved. TMRM fluorescence was excited at 543 nm (HeNe laser), and emission was measured at >560 nm. The positively charged lipophilic compound is sequestered into the mitochondria by virtue of their negative potential compared to the cytosol. Therefore, the changes in potential are reflected by changes in fluorescence intensity within the mitochondria which is measured after thresholding out the background signal from the cytosol to give a mean value per pixel within the mitochondrial population of a cell. It is important to emphasize that this measure is completely independent of mitochondrial mass or number in a cell.

Evaluation of the mitochondrial FAD level was done as described above for NADH.

All presented results are obtained from at least 5 coverslips and 2-3 different cell preparations.

### Results

### Evaluation of the Mitochondrial Membrane Potential (Δψm)

FIG. 1A shows representative results of an autofluorescence evaluation of NADH in primary myotubes after treatment with a selected DiSu+NAM(2) compositions, according to one or more examples of the present disclosure.

Treatment of primary myotube cultures loaded with TMRM with (1) DiSu dicholine succinate (DiSu, 50 μM), (2) nicotinamide (NAM; 20 μM) or (3) simultaneously with 50 μM DiSu and 20 μM NAM (ratio choline:succinate:nicotinamide=2:1:0.4) also referred to herein as DiSu+NAM(2) induced an increase in TMRM fluorescence compared to control. Subsequent application of the 1 μM FCCP induce complete mitochondrial depolarization and decrease the TMRM signal confirming ability of the compounds to increase mitochondrial membrane potential. The mixture of DiSu+NAM(2) (treatment 3) had the highest effect—21±2% (p<0.05; n=67 myotubes), compared to DiSu alone (treatment 1) and NAM alone (treatment 2).

FIG. 1C shows representative results of an evaluation of mitochondrial pool of NADH (measured as a difference Δ between FCCP and NaCN NADH autofluorescence) a after treatment a control, selected DiSu+NAM(2) compositions, and individual constituents, according to one or more examples of the present disclosure.

### Evaluation of the Mitochondrial NADH Pool

FIG. 1B shows representative results of a mitochondrial redox index in primary myotubes (which corresponds to NADH-dependent mitochondrial respiration) after treatment a control, selected DiSu+NAM(1) & DiSu+NAM(2) compositions, and individual constituents choline, succinate, and NAM.

NADH is produced in the Krebs cycle inside of mitochondria. It is used as a donor of electrons and substrate for the mitochondrial complex I. NADH is a fluorescent compound and this allows to measure its level in the mitochondria. However, NADH fluorescence cannot be distinguished from the fluorescent signal deriving from NADPH. To separate these two fluorescent signals, mitochondrial activators and inhibitors can be utilized. Accordingly, to measure the resting level of NADH autofluorescence, it was generated the “redox index,” a ratio of the maximally oxidized (response to 1 μM FCCP, a potent uncoupler of mitochondrial oxidative phosphorylation,—mitochondria respiring maximally with minimal level of NADH in mitochondria) and maximally reduced (response to 1 mM NaCN, a potent inducer of decrease in the mitochondrial membrane potential—both respiration and consumption of the NADH in mitochondria is blocked, which leads to the maximal values of NADH signals as shown in FIG. 1A.

Treatment of myotube cultures with two different blends of DiSu+NAM Blend 1 “Di Su+NAM(1)” 100 μM DiSu+40 μM NAM and Blend 2 “DiSu+NAM(2)” 50 μM DiSu+20 μM NAM (molar ratio choline:succinate:NAM=2:1:0.4), or with individual components used in the blends, e.g., choline (100 μM), succinate (50 μM), and NAM (50 μM), did not induce significant changes in the redox index (which corresponds to NADH-dependent mitochondrial respiration as shown in FIG. 1B.

FIG. 1C shows representative results of an evaluation of mitochondrial pool of NADH (measured as a difference Δ between FCCP and NaCN NADH autofluorescence) after treatment with a control, with selected DiSu+NAM compositions, and with individual components, according to one or more examples of the present disclosure.

It may be noted that both blends of DiSu and NAM, but not the individual components, significantly increased the mitochondrial pool of NADH (measured as a Δ between FCCP and NaCN NADH autofluorescence) as shown in FIG. 1C.

### Evaluation of Autophagy

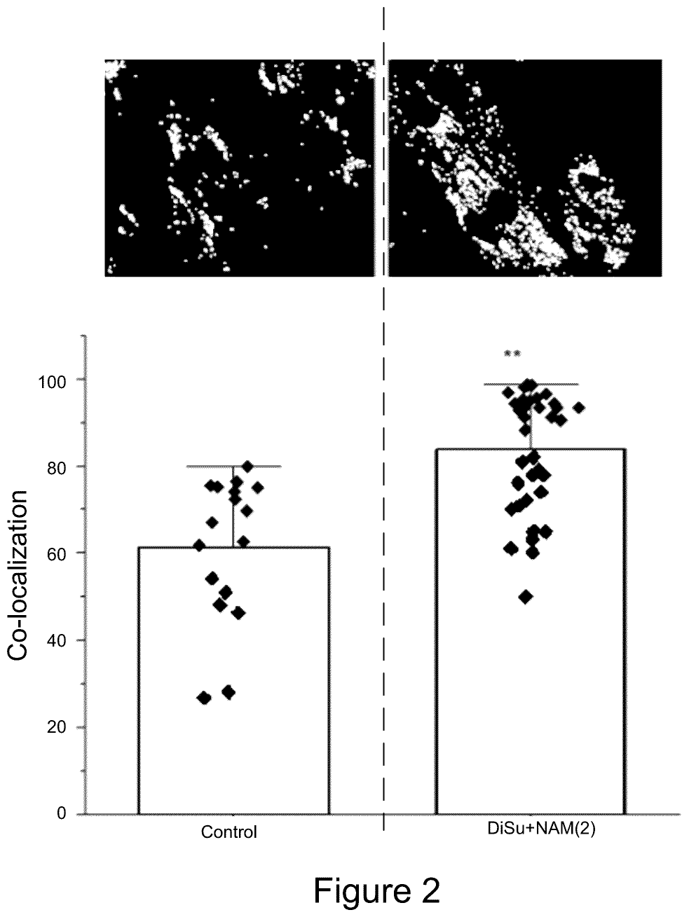

FIG. 2 shows representative results of an evaluation of autophagy in primary cultures of myotubes after treatment with a control and compare to after treatment with a selected DiSu+NAM composition evaluated as the level of co-localization of mitochondria and lysosomes, according to one or more examples of the present disclosure.

Mitophagy induced by treatments of primary myotubes either with Blend 2 DiSu+NAM(2) (50 μM DiSu+20 μM NAM (molar ratio choline:succinate:NAM=2:1:0.4) or individual compounds of the blend (DiSu, NAM, choline, succinate (all compounds are used in the molar amounts corresponding to the molar amounts of the compounds in the blend) was evaluated by analyzing the level of cellular co-localization of mitochondria (labelled with MitoTracker Green (green color)) and lysosomes (labeled with LysoTracker Red, red color) using confocal microscopy. Both DiSu and the blend of DiSu and NAM, but not NAM, choline bitartrate or succinate di-sodium salt, were potent to significantly increase autophagy in the treated cultures of primary myotubes. FIG. 2 demonstrates the results of cell treatment with the blend of DiSu and NAM e.g., DiSu+NAM(2) vs control (non-treated cells).

The following section describes evaluation of the FAD level results for Example 2.

### Evaluation of the FAD Level

FADH is essential for mitochondrial complex II and also a biological marker of the functionality of mitochondria.

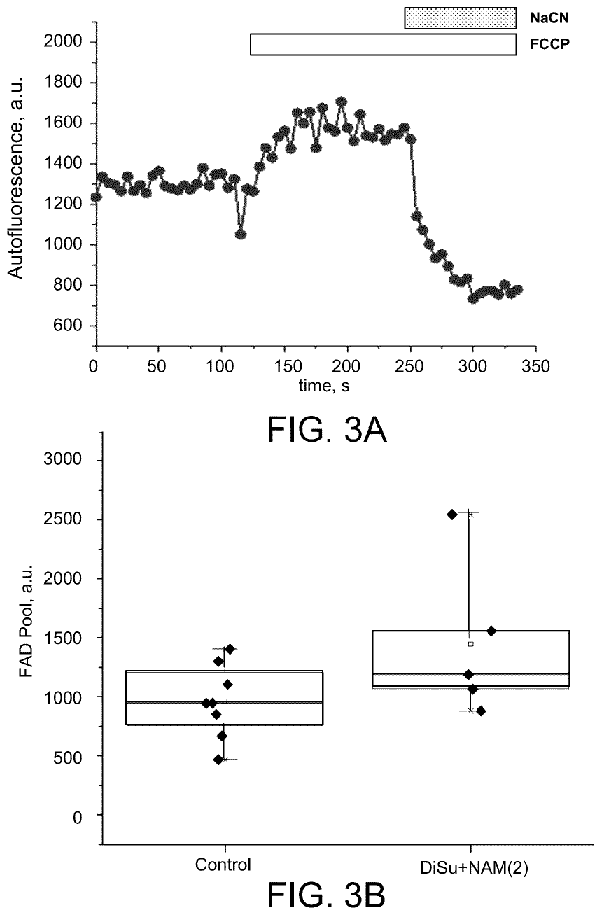

FIG. 3A shows representative results of evaluation of FAD autofluorescence in cultures of primary myotubes after treatment with a selected DiSu+NAM composition.

FAD++ is fluorescent and the mitochondrial level of FAD+++ was measured based on evaluation of its autofluorescence (in the same way as described above for NADH) as shown in FIG. 3A.

FIG. 3B shows representative results of an evaluation of mitochondrial pool of FAD after treatment with a control as compared to after treatment with a selected DiSu+NAM composition. Treatment of myotubes with a blend of DiSu and NAM DiSu+NAM(2) 50 μM DiSu+20 μM NAM (molar ratio choline:succinate:NAM=2:1:0.4), resulted in an increase of the mitochondrial pool of FAD++ as shown in FIG. 3B.

### Evaluation of the Overall Energy Capacity of the Activated Myotubes

The energy capacity of the cell is defined as the time between cessation of ATP production and the time of energetic collapse due to total ATP depletion and inability to maintain calcium homeostasis. Live-cell imaging of the fluorescent probe MagFura-2 was used to assess the energy capacity primary myotubes. Mg2+ is released from MgATP upon the hydrolysis of ATP, so the measurement of cellular free magnesium ([Mg2+]c) using the Mg2+-sensitive fluorescent probe MagFura-2 can be used as an indication of ATP consumption, for example using the protocol outline in Leyssens et al., (1996) J Physiol. 496, 111-128.

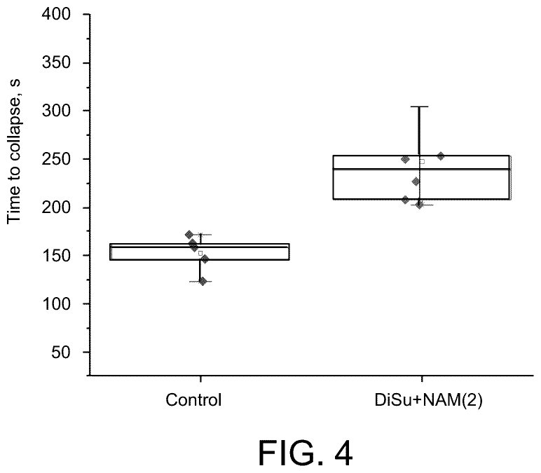

Application of inhibitors of glycolysis and/or oxidative phosphorylation blocks the ATP production in cells, which eventually leads to ATP depletion and subsequent Mg2+ release and an increase in Mag-Fura fluorescence as shown in FIG. 4. To estimate the overall ATP consumption, inhibitors of glycolysis (iodoacetic acid, 20 μm) and F1F0-ATP synthase (oligomycin, 2 μg/ml) were applied simultaneously.

FIG. 4 shows representative results of an evaluation of “time to collapse” of primary myotubes after treatment with a control as compared to after treatment with a selected DiSu+NAM(2) composition, according to one or more examples of the present disclosure;

In an example, the cultures were treated with 1 mM of caffeine to induce calcium dependent myotubes contractions to mimic muscle exercise, which leads to an increased consumption of ATP as shown in FIG. 4.

It is found that treatment of primary myotubes with a blend of 50 μM DiSu and 20 μM NAM; the molar ratio choline:succinate:NAM=2:1:0.4, significantly increases the time of contraction of myotubes to myotube collapse (from 147±7 to 238±9 min).

This surprising finding strongly suggests that simultaneous treatment of myotubes with DiSu and NAM increases the myotube energy capacity and thereby enhancing the capability of the muscle cells to sustain contraction for a longer period of time.

### Summary of Evaluation of the Production of ATP, NAD and FAD

Table 1 below summarizes results on the production of ATP, NADH and FAD in resting myocytes in response to treatment with different formulations of choline, succinate and nicotinamide or nicotinic acid. The levels of ATP, NADH and FAD in treated cells were normalized to the levels in control cells

TABLE 1

| Treatment of resting myocytes | ATP | NADH | FAD |
|---|---|---|---|
| Control (no treatment) | − | − | − |
| DiSu | ++ | ++ | + |
| Succinate* | + | − | − |
| NAM | + | + | − |
| Nicotinic acid | − | − | − |
| Choline** | + | − | − |
| DiSu + NAM | ++++++ | +++++ | +++++ |
| DiSu and Nicotinic acid | + | − | − |
| Succinate* and NAM | − | − | − |
| Choline** + Succinate + NAM | +++ | ++ | ++ |

*Sodium succinate (salt);
**Choline bitartrate

In all combined treatments with choline, succinate and NAM/nicotinic acid the molar ratio choline:succinate:NAM/nicotinic acid is 2:1:0.4

The Table 2 below summarizes results on the production of ATP, NADH and FAD in myocytes activated with different concentrations of caffein in response to treatment with different formulations of choline, succinate and nicotinamide or nicotinic acid. The levels of ATP, NADH and FAD in treated cells were normalized to the levels in control cells.

TABLE 2

| | 1 mM caffeine | | | 5 mM caffeine | | | 10 mM caffeine | | |
|---|---|---|---|---|---|---|---|---|---|
| Treatment | ATP | NADH | FAD | ATP | NADH | FAD | ATP | NADH | FAD |
| Control (no treatment) | − | − | − | − | − | − | − | − | − |
| DiSu | ++ | ++ | + | ++ | + | + | + | + | + |
| Succinate* | − | − | − | − | − | − | − | − | − |
| NAM | + | − | − | − | − | − | − | − | − |
| Nicotinic acid | − | − | − | − | − | − | − | − | − |
| Choline** | − | − | − | − | − | − | − | − | − |
| DiSu + NAM | +++++ | ++++ | ++++ | ++++ | ++++ | ++++ | +++ | +++ | ++ |
| DiSu + Nicotinic acid | + | + | − | − | + | + | + | − | − |
| Succinate* + NAM | + | − | − | − | − | − | − | − | − |
| Choline** + Succinate* + NAM | +++ | ++ | ++ | ++ | + | + | ++ | + | + |

*Sodium succinate (salt);
**Choline bitartrate

In all combined treatments with choline, succinate and NAM/nicotinic acid the molar ratio choline:succinate:NAM/nicotinic acid is 2:1:0.4

The results presented in Table 1 and Table 2 show that:

Treatment of resting myocytes with the composition consisting of DiSu and NAM (molar ratio choline:succinate:NAM=2:1:0.4) results in a significant increase in intracellular levels of both ATP, NADH and FAD compared to the control (p<0.05). The effect of the composition was approximately 1.5-fold greater than sum of the effects of the individual components, DiSu and NAM, respectively. This indicates that the compounds of the composition of the present disclosure works in synergy improving production of ATP, NADH and FAD production in resting myocytes; and

Treatment of activated myocytes with the composition consisting of DiSu and NAM (molar ratio choline:succinate:NAM =2:1:0.4) results in a significant increase in intracellular levels of both ATP, NADH and FAD compared to the control (p<0.05) both, in 1 mM, 5 mM and 10 mM caffein activated cultures. Compared to results of treatment with the single compounds of the composition, the effect is synergetic.

Accordingly, compositions of the present disclosure consisting essentially of choline cation, succinate anion and NAM in the molar ratio about 2:1:04 are effective for the enhancement of production of ATP, NADH and FAD in both resting and activated myocytes in vitro.

According to the present disclosure, compositions where the molar ratio choline:succinate:NAM is within the range of 0.5-3:3-0.5:0.01-10 are effective for enhancing the generation of energy molecules NAD, FAD and ATP in mitochondria and enhancing autophagy in primary cultures of muscle cells. Therefore, the results presented in Tables 1 and 2 are representative and not limiting to the scope of the disclosure to compositions with the molar ratio choline:succinate:NAM of 2:1:0.4.

### Example 3

### Study of Skeletal Muscle Function and Endurance Performance in Athletes

### Brief Description of the Study

A randomized, double-blind, placebo-controlled study enrolling at least 36 (18 Elite and 18 Sub-Elite trained endurance male runners. 18 placebo and 18 Beverage-3 are given as a daily oral dose for 4-weeks.

### Study Details:

| | |
|---|---|
| Allocation: | Randomized |
| Intervention Model: | Parallel Assignment |
| Masking: | Triple (Participant, Investigator, Outcomes |
| | Assessor) |
| Primary Purpose: | Testing the effect of Beverage-3 |
| Active Comparator: | Dietary Supplement: Beverage-3 (Example 1) |
| | 1 dose of Beverage-3 to be taken daily. |
| Placebo Comparator: | Dietary Supplement: Placebo beverage (excipients |
| | containing beverage) 1 dose of Placebo beverage |
| | to be taken daily |

### Eligibility Criteria

### Inclusion Criteria:

1. Trained elite and sub-elite male runners, age between 18-40 years;
2. Participants will be running>100 km/week;
3. Elite participants are required to have a 3,000 m running personal best time below 9:00 (mm:ss), and/or a VO2max result greater than 65 ml·kg−1·min−1; and
4. The sub-elite cohort is required to have a 3,000 m running personal best faster than 10:00 min and/or a VO2max>60 ml·kg−1·min−1.

### Exclusion Criteria:

1. Human subjects with diagnosed medical conditions involving thyroid function or other chronic disturbances of metabolic rate; or
2. Human subjects who are unable to complete the training or testing protocols.
   Outcome Measures (Time Frame: 4 weeks)

### Primary Outcome Measures:

1. Change in plasma levels over time of creatine kinase (CK); and
2. Change in race performance time during a 3000 m track race in elite runners.

### Secondary Outcome Measures:

1. Change from baseline in aerobic capacity (VO2max);
2. Change from baseline in running economy via indirect calorimetry;
3. Change from baseline in lean body mass via Dual-energy X-ray Absorptiometry (DXA);
4. Change from baseline in body fat mass via Dual-energy X-ray Absorptiometry (DXA);
5. Change from baseline in Resting Metabolic Rate (RMR);
6. Change from baseline in maximal muscle strength during 1-repetition maximum leg press;
7. Change in iron absorption and hemoglobin mass via determination of carboxyhemoglobin (percent HbCO) (sub-elite runners only);
8. Change in acylcarnitines levels via metabolomics in plasma;
9. Change in mitochondrial function via respirometry in muscle biopsies (sub-elite runners only);
10. Change in mitochondrial gene expression via RNA-seq in muscle biopsies (sub-elite runners only);
11. Change in plasma levels of Urolithin A; and
12. Change in plasma levels of inflammatory marker CRP.

### Example 4

### Study of Bioenergetics and Muscle Function Improvement in Elderly Skeletal Muscle

### Summary:

A randomized, double-blind, single-center, placebo-controlled trial enrolling 50-60 healthy elderly human subjects (33 placebo and 33 Beverage-1 administration) who are ≥65 and ≤90 years of age with evidence of low mitochondrial function. Beverage-1 or Placebo are orally administered for 4 months.

### Study Details:

| | |
|---|---|
| Allocation: | Randomized |
| Intervention Model: | Parallel Assignment |
| Masking: | Triple (Participant, Investigator, Outcomes |
| | Assessor) |
| Primary Purpose: | Testing the effect of Beverage-3 |
| Active Comparator: | Dietary Supplement: Beverage-3 (Example 1) |
| | 1 dose of Beverage-3 to be taken daily. |
| Placebo Comparator: | Dietary Supplement: Placebo beverage (excipients |
| | containing beverage) 1 dose of Placebo beverage |
| | to be taken daily |

### Eligibility Criteria:

1. Adult human volunteers of all sexes ≥65 and ≤90 years of age.
2. Capable of walking 6 minute walk distance of <550 meters.
3. Have ATP max<1 mM/sec (in the hand FDI muscle).

### Exclusion Criteria:

1. Human subjects who have significant disease(s) or condition(s) which, in the opinion of the investigator, may put the human subject at risk because of their participation in the trial or may influence either the results of the trial or the human subject's ability to participate in the trial;
2. Hospitalization within 3 months for major atherosclerotic events (defined as combined incidence of myocardial infarction, urgent target-vessel revascularization, coronary bypass surgery and stroke) and for any hospitalization within 2 months;
3. Have any metal implants in the right limbs, including non-MRI compatible metal stents, titanium pins/markers, etc.;
4. Have an implanted cardiac pacemaker or other implanted non-MRI compatible cardiac device;
5. Chronic, uncontrolled hypertension as judged by the Investigator (e.g., Baseline SBP>150 mm Hg, DBP>90 mm Hg) or a SBP>150 mm Hg or DBP>95 mm Hg at the time of screening or baseline; If the initial BP reading is above these values, the reading may be repeated one time within 20 minutes of the initial reading;
6. Body mass index<18 or >32 kg/m2;
7. Severe chronic kidney disease requiring treatment with hemodialysis or peritoneal dialysis;
8. Additional laboratory abnormalities determined as clinically significant by the Investigator;
9. Clinically significant abnormalities on physical examination (as judged by the Investigator);
10. Clinically significant and chronic uncontrolled renal, hepatic, pulmonary, endocrine, neurologic disorders, bone, or gastrointestinal system dysfunction;
11. History of seizures or epilepsy;
12. History of serious mental illness as judged by the Investigator;
13. Oral temperature>37;5° C. at the time of the physical;
14. Suspicion, or recent history, of alcohol or substance abuse or tobacco use
15. Human subjects who in the opinion of the Investigator have a clinically significant abnormal 12-lead ECG during the screening period, presence of atrial fibrillation, varying degrees of AV block, existence of a left bundle branch block, or evidence of previous myocardial infarction;
17. Human subjects who are either unwilling to agree to refrain from using or are found to be using supplementary antioxidant vitamins (e.g., Coenzyme Q10, resveratrol, L-carnitine) from 7 days prior to dosing and throughout the treatment period;
18. Human subjects who are either unwilling to agree to refrain from using or are found to be using the following dietary restrictions (pomegranate juice, walnuts, pecans, strawberry, raspberry blackberry) from 7 days prior to dosing and throughout the treatment period;
19. Are currently enrolled in a clinical trial involving an investigational product or non-approved use of a drug or device or concurrently enrolled in any other type of medical research judged not to be scientifically or medically compatible with this study; or
20. Have participated within the last 30 days in a clinical trial involving an investigational product (if the previous investigational product has a long half-life, 3 months or 5 half-lives, whichever is longer, should have passed.

### Primary Outcome Measures: (Time Frame: 4 Months)

1. Change in 6 minute walking distance (6MWD) at the end of study intervention compared to baseline; and
2. Percent change from baseline in ATP max (maximal ATP synthesis rate) in hand skeletal muscle (via Magnetic Resonance Spectroscopy).

### Secondary Outcome Measures (Time Frame: 4 Months):

1. Percent change from baseline in contraction number during a hand muscle fatigue test;
2. Percent change from baseline in ATP max (maximum ATP synthesis rate) in leg skeletal muscle (via MRS);
3. Percent change from baseline in contraction number during a leg muscle fatigue test;
4. Change in Short Physical Performance Battery (SPPB) scores at the end of study intervention compared to baseline;
5. Change in exercise tolerance compared to baseline (via cycle ergometry);
6. Change in hand grip strength at the end of study intervention compared to baseline;
7. Change in leg muscle strength (1-RM and 10-RM) at the end of study intervention compared to baseline;
8. Change in muscle size (cross-sectional area of the muscles) at the end of study intervention compared to baseline;
9. Change in mitochondrial function on muscle biopsy samples at the end of study intervention compared to baseline (via respirometry);
10. Effect of Beverage-1 on mitochondrial gene and protein expression in muscle tissue;
11. Effect of Beverage-1 on plasma acylcarnitines;
12. Effect of Beverage-1 on quality of life questionnaire (SF36);
13. Change from baseline in plasma lipid profile; and
14. Change from baseline in plasma for circulating biomarkers (myostatin, follistatin).

### Example 5

### Assessment of the Effect of Compositions of the Invention on Metabolism of Skeletal Muscle Cells Using Sarcopenia an In Vitro Cell Model and an In Vivo Aging Animal Model

Sarcopenia is a progressive and generalized skeletal muscle disorder involving the accelerated loss of muscle mass and function that is associated with increased adverse outcomes, including falls, functional decline, frailty, and mortality. Although sarcopenia is recognized as a disease with an International Classification of Diseases code, there is a lack of consensus with regard to its clinical identification. Its multifactorial pathogenesis includes enhanced expression of muscle growth inhibitors and oxidative stress, neuromuscular junction dysfunction, impaired function of muscle stem cells (MuSCs), reduced mitochondrial biogenesis and function, diminished muscle protein synthesis, activation of catabolic pathways, and development of insulin resistance.

The result of this is skeletal muscle atrophy which is characterized by a decrease in muscle fiber size due to a decreased protein synthesis and deficient ATP synthesis and utilization, which leads to degradation of contractile muscle fibers. Sarcopenia can occur after denervation and immobilization, and, also due to age-related changes in skeletal muscle energy metabolism. Glucocorticoids (GCs) may also increase protein breakdown contributing to the loss of muscle mass and myofibrillar proteins. GCs, in particular dexamethasone, may be used in vitro to induce atrophic conditions.

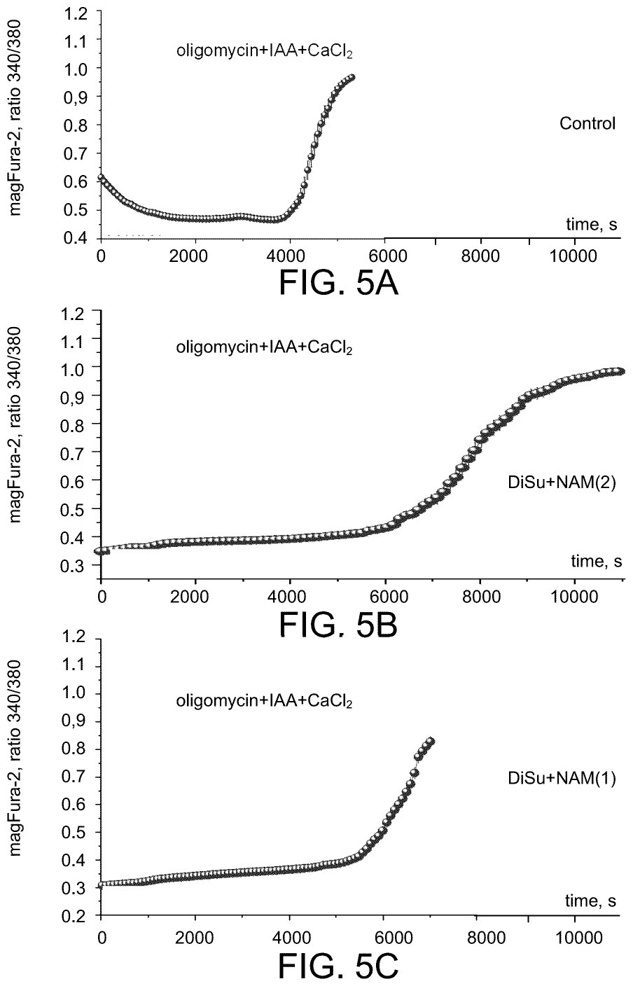

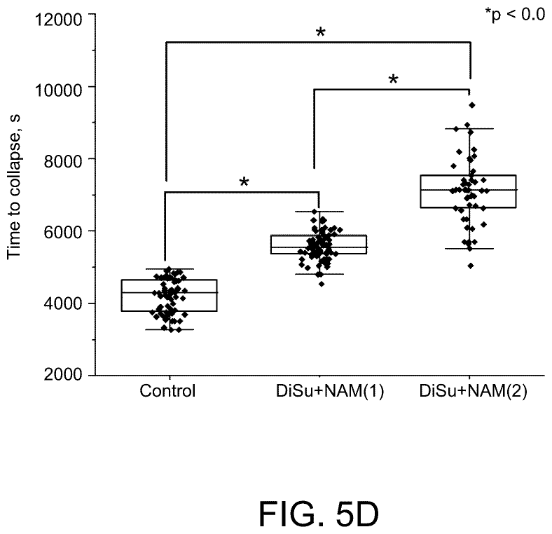

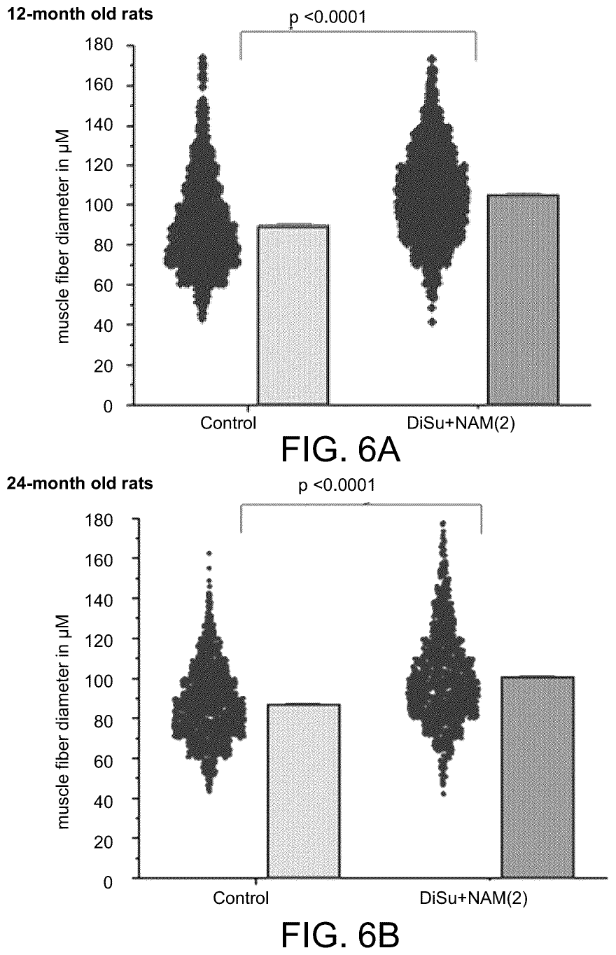

The results presented below show the effect of compositions of the present human subject matter on energy capacity of dexamethasone-treated primary myotubes in vitro as shown in example results depicted in FIGS. 5A-5D Similarly FIGS. 6A and 6B show representative results of an in vivo evaluation of maintenance of skeletal muscle mass in vivo using the age-related sarcopenia animal model as shown in the example results depicted in the charts shown in FIGS. 6A and 6B.

### Effect of Compositions of the Invention on Energy Capacity of Primary Myotubes Using Dexamethasone-Induced Sarcopenia Cell Culture Model

Primary rat myocyte cultures were prepared as described by Yao G., et al. ((Yao G., et. al. (2011) J Cell Sci v.124: 4194-4202. doi: 10.1242/jcs.088260).

Briefly, to prepare mixed cultures of skeletal myocytes and fibroblast cells, hind legs of postnatal day 2-5 pups were taken and skins and fat tissues were carefully removed The tissues was then digested in 0.2% collagenase solution at 37° C. for 30-40 minutes. The reaction will be stopped by adding 2-3 ml FBS and the tissue suspension was centrifuged at 2010 rpm for 10 minutes.

The cell pellet was resuspended in pre-warmed growth medium (DMEM containing 20% FBS, 100 I.U./ml penicillin and 200 I.U./ml streptomycin) and plated on 22 mm coverslips pre-coated with 0.2% gelatin for 1 hour at 37° C. Cells were then cultured in a humidified CO2 incubator (5% CO2 in air) at 37□C. After 2-3 days the medium was changed to a low serum medium (DMEM containing 10% FBS without antibiotics) and medium was changed every 3-4 days. All experiments were performed with 5-11 days cultured cells.

To test energetic capacity of cultures myotubes in atrophic conditions, myocyte cultures were differentiated for 5 days followed incubation in the presence of 1, 10, and 100 μM dexamethasone for 24 h. Dexamethasone-treated primary cultures were then loaded with magnesium selective fluorescent indicator MagFura-2. Cells were divided in the following groups—control vs the following treatment groups:
no treatment (control)
DiSu+NAM (2) (50 μM DISU+20 μM NAM (molar ratio choline:succinate:NAM=2:1:0.4);
DiSu+NAM (1) (100 μM DISU+20 μM NAM (molar ratio choline:succinate:NAM=2:1:0.2);
DiSu (50 μM)
NAM (20 μM)
Choline* (50 μM choline chloride)
Succinate* (50 μM disodium succinate)
Choline*+succinate*+NAM (molar ratio choline:succinate:NAM=2:1:0.4)
Creatine (10 mM)
DiSu+NAM (2)+creatine
DiSu (50 μM)+creatine (10 mM)

All cultures were treated with an inhibitor of the ATP production in cellular cytoplasm via glycolysis, Iodoacetic Acid (IAA) (20 μM), and an inhibitor of the ATP production in mitochondria by oxidative phosphorylation, oligomycin (2 μg/ml). These treatments lead to non-compensated consumption of ATP (e.g., APT is not produced while it is used by cells) resulting in ATP decrease and increase in Mg2+ level. Energy collapse of cells also leads to a massive increase in intracellular calcium. The cellular concentration of both cations can be indirectly measured by measuring fluorescence of Mag Fura-2 dye. Accordingly, the cells in all cultures were also loaded with fluorescent Mag Fura-2 dye that has affinity to both cations to measure the release of intracellular Mg2+ and Ca2+ cations following the treatment with the ATP synthesis inhibitors.

As expected, the intracellular concentration of released Mg2+ and Ca2+ cations in the inhibitor treated control cultures increased very sharply, indicating a rapid exhaustion of cellular energy deports and cell collapse.

However, DiSu, creatine, DiSu+creatine, DiSu+NAM (2), DiSu+NAM (1), and DiSu+NAM ((2) or (1))+creatine treatments, all extended the time until cell total energy exhaustion and collapse, yet to a different extent. The potency of the later compounds to preserve cells from energy exhaustion and extending time to collapse (e.g., time to death) were as the following: DiSu+NAM(2)+creatine>DiSu+NAM (3)+creatine>DiSu+NAM(3)>DiSu+NAM (ii)≈DiSu+creatine>DiSU>>creatine. Neither choline or succinate or NAM treatments had a noticeable effect on the energy capacity of the cells compared to control cells. The treatment of the cells with choline chloride combined with disodium succinate and NAM (choline:succinate:NAM=2:1:0.4) resulted on a slowing of the energy depletion compared to the control cells. The time until cell collapse in these cultures was comparable to cultures treated with DiSu alone however, it was much shorter than in cultures treated with DiSu+NAM ((2) or (3)) or creatine combined treatments.

Results of the DiSu+NAM ((2) and (1)) treatment vs control treatment is presented in FIGS. 5A, 5B and 5C.

FIGS. 5A, 5B and 5C presents the results of evaluation of energetic capability of primary rat myotubes treated dexamethasone to induce atrophy, and further treated with oxidative phosphorylation and glycolysis inhibitors (oligomycin and IAA, correspondingly) to block ATP synthesis in the cells. An increase in intracellular concentrations of Mg and Ca cations is released in response to the inhibitor treatment. and B—the myotube energetic capability evaluated as time to myotube death (i.e. time to collapse) in the presence or absence of DISU+NAM.

FIG. 5A shows representative results of a fluorescence evaluation of energy capacity of primary myotubes using a dexamethasone-induced sarcopenia cell culture model in which the inhibitor treated cells were maintained without any additional treatment (control).

FIG. 5B shows representative results of a fluorescence evaluation of energy capacity of primary myotubes using a dexamethasone-induced sarcopenia cell culture model after treatment with a selected DiSu+NAM composition. The primary myotubes were treated with a blend of 50 μM DiSu and 20 μM NAM in which the molar ratio choline:succinate:NAM is 2:1:0.4 (DiSu+NAM(2)).

FIG. 5C shows representative results of a fluorescence evaluation of energy capacity of primary myotubes using a dexamethasone-induced sarcopenia cell culture model after treatment with another selected DiSu+NAM composition, according to one or more examples of the present disclosure. In FIG. 5C, the blend of DiSu and NAM is 100 μM DiSu and 20 μM NAM, in which the molar ratio choline:succinate:NAM is 2:1:0.2 (DiSu+NAM(1)).

FIG. 5D shows representative results of an evaluation of “time to collapse” of primary myotubes after no treatment as compared to after treatment with a selected DiSu+NAM(2) composition, according to one or more examples of the present disclosure.

As shown in FIG. 5D, the myotube energetic capability evaluated as time to myotube death (i.e., time to collapse) in the presence or absence of DiSu+NAM. The shortest time to collapse (between about 3200 to about 4900 s) is observed in the leftmost plot labeled “control.”

Consistent with the graph shown in FIG. 5B, the time to collapse for the tested blend 50 μM DiSu and 20 μM NAM (molar ratio choline:succinate:NAM is 2:1:0.4) DiSu+NAM(2) is longest (between about 5500 to about 9000 s) as shown on the rightmost plot of FIG. 5D.

The center plot of FIG. 5D shows that the time to collapse for the tested blend 100 μM DiSu and 20 μM NAM (molar ratio choline:succinate:NAM is 2:1:0.2) DiSu+NAM(1) is longer (between about 4800 to about 6500 s) than the test with the control.

### Conclusion

This study results demonstrate that a combination of choline cation with divalent succinate anion in the molar ratio 2:1, alone, such as the dicholine salt of succinic acid (DiSu), can increase the general energetic capacity of skeletal muscle cells affected by myotube atrophy induced by dexamethasone and help the cells to survive without energy supply for longer.

This muscle cell life-saving effect can further be significantly enhanced by a combination of DiSu with either creatine, which is a natural storage of ATP in cytosol of energy-demanding body cells, like muscle and brain cells, that is released immediately when needed, and/or further with NAM, which works in synergy with DiSu to generate ATP via oxidative phosphorylation, e.g., as DiSu+creatine or DiSu+NAM+creatine.

### Example 6

### Effect of Compositions of the Invention on Skeletal Muscle Cells In Vivo

The effect of DiSu+NAM composition on skeletal muscle was further studied in aging rats in vivo.

Four groups of rats as shown below were used in this study. Two of the four groups received treatment with DiSu+NAM(2) (50 μM DiSu and 20 μM NAM; molar ratio choline:succinate:NAM=2:1:0.4) and two other groups received no treatment and are labelled “control”).

In FIG. 6A, the results are shown for:
control group A (Wistar male rats (n=3), aged 12 months); and
DiSu+NAM(2) group A (Wistar male rats (n=3), aged 12 months).

In FIG. 6B, the results are shown for:
control group B (Wistar male rats (n=3), aged 24 months);
DiSu+NAM(2) group B (Wistar male rats (n=3), aged 24 months).

The animals were drinking water without DiSu+NAM(2) (control groups A and B) or water with added 50 mg/kg (animal weight) DiSu+NAM(2) for 7 consequent days. On day 8 the animals were sacrificed and skeletal muscle tissue from hind legs were taken to prepare acute slices. The slices were loaded with fluorescent indicator fluo-4. Fluorescent images were taken using confocal microscope Zeiss LSM 900. The diameter of myotubes was calculated for each group (15 slices per group measuring multiple myotubes per slice) using Zeiss software.

The results of evaluation of the thickness of microtubes is presented in FIGS. 6A and 6B. FIG. 6A shows that a relatively short-term supplementation (7 days) with DiSu+NAM(2) increased the myotube diameter in young animals. Surprisingly, FIG. 6B shows a similar increase in myotube diameter in aging animals. Notably it was observed that an average diameter of myotubes was different in the two control groups: 90.2±0.5 μM in 12 months old rats of the control group A versus 87.2±0.5 μM in 24 months old rats of the control group B;

Thus, a supplementation of rats with DiSu+NAM(2) for 7 consequent days significantly increased the diameter of myotubes in both DiSu+NAM(2) groups: in the treatment group A the diameter was increased to 105.6±0.6 μM, and in the treatment group B—to 100.7±0.6 μM.

### Conclusion

This study demonstrates that a dietary supplementation with DiSu, especially combined with NAM and/or creatine, is beneficial for increasing skeletal muscle fiber thickness, and, consequently, skeletal muscle mass in both young and aging animals. Notably, the skeletal muscle tissue of aging and young animals practically equally benefits from the treatment (an increase in thickness of the skeletal muscle fibers by about 15% was observed in both treatment groups), but the aging animals benefit is remarkable in that the skeletal tissue mass was regained to the level of normal (e.g., non-treated) young animals.

Accordingly, results of this study indicate that a dietary supplement with DiSu (alone or with NAM and/or creatine added), which enhances mitochondrial synthesis of ATP by supplying substrates of oxidative phosphorylation in mitochondria, rejuvenates the cellular pool of mitochondria by enhancing mitophagy, and increases the total energy capacity of skeletal muscle cells, may reduce or delay the onset of skeletal muscle atrophy related to aging, or recover the volume of already lost muscle tissue in aging people.

In summary, the representative examples of compositions disclosed herein, illustrate by way of working examples that the disclosed method of formulating a composition to contain a predetermined dosage amount of choline and succinate in a molar ratio of choline to succinate of 2:1 is effective to reduce a frequency and/or a severity of one or more symptoms selected from skeletal muscle weakness, skeletal muscle pain, low skeletal muscle tone, physical exercise intolerance, lack of muscular endurance, and loss of skeletal muscle mass in a human subject consuming the composition during an initial course of treatment and is also effective to increase skeletal muscle mass a human subject consuming the composition during an initial course of treatment. Accordingly, providing to the human subject, the composition containing the predetermined dosage amount of choline and succinate in the molar ratio of choline to succinate of 2:1, alone or in combination with one or more selected NAM components and/or one or more selected creatine components is effective to provide skeletal muscle benefits not found in existing compositions or methods.

What is claimed is:

1. A method for treating one or more symptoms in a human subject experiencing an abnormal skeletal muscle condition, the method comprising:
formulating a composition to contain a predetermined dosage amount of choline and succinate in a molar ratio of choline to succinate of 2:1 that is effective to reduce a frequency and/or a severity of one or more symptoms selected from skeletal muscle weakness, skeletal muscle pain, low skeletal muscle tone, physical exercise intolerance, lack of muscular endurance, and loss of skeletal muscle mass in a human subject consuming the composition during an initial course of treatment; and
providing to the human subject, the composition containing the predetermined dosage amount of choline and succinate in the molar ratio of choline to succinate of 2:1.

2. The method of claim 1, further comprising providing the predetermined dosage amount of the composition for consumption by the human subject in a selected dosage form that includes from about 100 mg to about 1000 mg of choline and succinate in a molar ratio of choline to succinate of 2:1 per serving in one or more servings up to a total of about 5000 mg per day.

3. The method of claim 1, further comprising, formulating the composition to further comprise:
at least one nicotinamide component selected from nicotinamide, nicotinamide riboside, and/or nicotinamide mononucleotide.

4. The method of claim 3, further comprising formulating the molar ratio of the choline to the succinate to the at least one nicotinamide component in the composition to be about 2:1:0.01-10.

5. The method of claim 1, further comprising formulating the composition to further comprise at least one creatine component selected from creatine, arginine, and/or glycine.

6. The method of claim 2, further comprising providing an initial treatment course that includes at least a five to seven day supply of the composition in the selected dosage form.

7. The method of claim 2, further comprising formulating the selected dosage form of the composition as a component of a medical food product or a medical beverage product.

8. The method of claim 2, further comprising the selected dosage form of the composition as a component of a nutritional product.

9. The method of claim 1, wherein the human subject is an aging human over 35 years old.

10. The method of claim 1, wherein the abnormal skeletal muscle condition of the human subject is induced by a physical injury, a psychological factor, or an environmental factor.

11. The method of claim 1, wherein the abnormal skeletal muscle condition is selected from muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, and/or sporadic inclusion body myositis.

12. A method for increasing skeletal muscle mass in a human subject, the method comprising:
formulating a composition to contain a predetermined dosage amount of choline and succinate in a molar ratio of choline to succinate of 2:1 that is effective to increase skeletal muscle mass a human subject consuming the composition during an initial course of treatment; and
providing to the human subject, the composition containing the predetermined dosage amount of choline and succinate in the molar ratio of choline to succinate of 2:1.

13. The method of claim 12, further comprising providing the predetermined dosage amount of the composition for consumption by the human subject in a selected dosage form that includes from about 100 mg to about 1000 mg per serving in one or more servings up to a total of about 5000 mg per day.

14. The method of claim 13, further comprising providing an initial treatment course that includes at least a five to seven day supply of the composition in the selected dosage form.

15. The method of claim 13, further comprising formulating the composition to further comprise at least one nicotinamide component selected from nicotinamide, nicotinamide riboside, and/or nicotinamide mononucleotide.

16. The method of claim 15, further comprising further the molar ratio of the choline to the succinate to the at least one nicotinamide component in the composition to be about 2:1:0.01-10.

17. The method of claim 13, further comprising formulating the composition to further comprise at least one creatine component selected from creatine, arginine, and/or glycine.

18. The method of claim 13, further comprising providing the composition as a sports beverage for consumption by a healthy human subject, a physically active human subject, and/or a person engaged in sports.

19. The method of claim 14, further comprising formulating the dosage form of the composition as a component of a nutritional beverage or a sports beverage.

20. The method of claim 14, further comprising formulating the dosage form of the composition for consumption by an aging human subject, or a human subject affected by a skeletal muscle loss as a symptom of a musculoskeletal disease or a musculoskeletal disorder selected from muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related sarcopenia, age-related muscle-wasting, physical fatigue, muscle fatigue, inclusion body myositis, sporadic inclusion body myositis.
